SEQUENCE LISTING (47 sequences)

SEQ ID NO: 1 - DNA, 1161 bp, Artificial Sequence
  Description of Artificial Sequenceclone PIP-1 improved pentin lipid acyl hydrolase
atgggatccgcattttccacacaagcgaaagcctctaaagatggaaacttagtcacagttcttgccattgatggaggtggtatcagaggaattatccccggagttattctcaaacaactagaagctactcttcagagatgggactcaagtgcaagactagcagagtattttgatgtggttgccgggacgagcactggagggattataactgccattctaactgccccggacccacaaaacaaggaccgtcctttgtatgctgccgaagaaattatcgacttctacatagagcatggtccttccatttttaataaatccaccgcctgctcgttgcctggtatcttttgtccaaagtatgatgggaagtatttacaagaaataataagccagaaattgaatgaaacactactagaccagacaacaacaaatgttgttatcccttccttcgacatcaagcttcttcgtccaaccatattctcaactttcaagttagaggaagttcctgagttaaatgtcaaactctccgatgtatgcatgggaacttcagcagcaccaatcgtatttcctccctattatttcaagcatggagatactgaattcaatctcgttgatggtgcaatcatcgctggtattccggccccggttgctctcagcgaggtgctccagcaagaaaaatacaagaataaagaaatccttttgctgtctataggaactggagttgtaaaacctggtgagggttattctgctaatcgtacttggactattttcgattggagtagtgaaactttaatcgggcttatgagtcatggaacgagagccatgtctgattattacgttggctcacatttcaaagcccttcaaccccagaataactacctccgaattcaggaatacgatttagatccggcactggaaagcattgatgatgcttcaacggaaaacatggagaatctggaaaaggtaggacagagtttgttgaacgaaccagttaaaaggatgaatctgaatacttttgtcgttgaagaaacaggtgaaggtaccaatgcagaagctttagacaggctggctcagattctttatgaagaaaagattactcgtggtctcggaaagatatctttggaagtggataacattgatccatatactgaacgtgttaggtaa

SEQ ID NO: 2 - protein, 386 aa, Artificial Sequence
  Description of Artificial Sequenceclone PIP-1 improved pentin lipid acyl hydrolase
MGSAFSTQAKASKDGNLVTVLAIDGGGIRGIIPGVILKQLEATLQRWDSSARLAEYFDVVAGTSTGGIITAILTAPDPQNKDRPLYAAEEIIDFYIEHGPSIFNKSTACSLPGIFCPKYDGKYLQEIISQKLNETLLDQTTTNVVIPSFDIKLLRPTIFSTFKLEEVPELNVKLSDVCMGTSAAPIVFPPYYFKHGDTEFNLVDGAIIAGIPAPVALSEVLQQEKYKNKEILLLSIGTGVVKPGEGYSANRTWTIFDWSSETLIGLMSHGTRAMSDYYVGSHFKALQPQNNYLRIQEYDLDPALESIDDASTENMENLEKVGQSLLNEPVKRMNLNTFVVEETGEGTNAEALDRLAQILYEEKITRGLGKISLEVDNIDPYTERVR

SEQ ID NO: 3 - DNA, 1170 bp, Artificial Sequence
  Description of Artificial Sequenceclone PIP-2 improved pentin lipid acyl hydrolase
atgggatccgcattttccacacaagcgaaagcttctaaagatggaaacttagtcacagttcttgccattgatggaggtggtatcagaggaattatccccggagttattctcaaacaactagaagctactcttcagagatgggactcaagtgcaagactagcagagtattttgatgtggttgctgggacgagcactggagggattataactgccattctaactgccccggacccacaaaacaaggaccgtcctttgtatgctgccgaagaaattatcgacttctacatagagcatggtccttccatttttaataaatccaccgcctgctcgttgcctggtatcttttgtccaaagtatgatgggaagtatttacaagaaataataagccagaaattgaatgaaacactactagaccagacaacaacaaatgttgttatcccttccttcgacatcaagcttcttcgtccaaccatattctcaactttcaagttagaggaagctcctgagttaaatgtcaaactctccgatgtatgcatgggaacttcagcagcaccaatcgtatttcctccctattatttcaagcatggagatactgaattcaatctcgttgatggtgcaatcgtcgctgatattccggccccggttgctctcagcgaggtgctccggcaagaaaaatacaagaataaagaaatccttttgctgtctataggaactggagttgtaaaacctggtgagggttattctgctaatcgtacttggactattttcgattggagtagtgaaactttaatcgggcttatgggtcatggaacgagagccatgtctgattattacgttggctcacatttcaaagcccttcaaccccagaataactacctccgaattcaggaatacgatttagatccggcactggaaagcattgatgatgcttcaacggagaacatggagaatctggaaaaggtaggacagagtttgttggacgaaccagttaaaaggatgaatctgaatacttttgtcgttgaagaaacaggtgaaggtaccaatgcagaagctttagacaggctggctcggattctttatgaagaaaagattactcgtggtctcggaaagatatctttggaagtggataacattgatccatatactgaacgtgttaggaaactgctattc

SEQ ID NO: 4 - protein, 390 aa, Artificial Sequence
  Description of Artificial Sequenceclone PIP-2 improved pentin lipid acyl hydrolase
MGSAFSTQAKASKDGNLVTVLAIDGGGIRGIIPGVILKQLEATLQRWDSSARLAEYFDVVAGTSTGGIITAILTAPDPQNKDRPLYAAEEIIDFYIEHGPSIFNKSTACSLPGIFCPKYDGKYLQEIISQKLNETLLDQTTTNVVIPSFDIKLLRPTIFSTFKLEEAPELNVKLSDVCMGTSAAPIVFPPYYFKHGDTEFNLVDGAIVADIPAPVALSEVLRQEKYKNKEILLLSIGTGVVKPGEGYSANRTWTIFDWSSETLIGLMGHGTRAMSDYYVGSHFKALQPQNNYLRIQEYDLDPALESIDDASTENMENLEKVGQSLLDEPVKRMNLNTFVVEETGEGTNAEALDRLARILYEEKITRGLGKISLEVDNIDPYTERVRKLLF

SEQ ID NO: 5 - DNA, 1170 bp, Artificial Sequence
  Description of Artificial Sequenceclone PIP-3 improved pentin lipid acyl hydrolase
atgggatccgcattttccacacaagcgaaagcttctaaagatggaaacttagtcacagttcttgccattgatggaggtggtatcagaggaattatccccggagttattctcaaacaactagaagctactcttcagagatgggacccaagtgcaagactagcagagtatttcgatgtggttgccgggacgagcactggagggattatagctgccattctaactgccccggaccctcaaaacaaggaccgtcctttgtatgctgccgaagaaattatcgacttctacatagagcatggtccttccatttttaataaatccaccgcctgcccgttgcctggtatcttttgtccaaagtatgatgggaagtatttacaagaaataataagccagaaattgaatgaaacactactagaccagacaacaacaaatgttgttatcccttccttcgacatcaagcttcttcgtccaaccatattctcaactttcaagttagaggaagttcctgagttaaatgtcaaactctccgatgtatgcatgggaacttcagcagcgccaatcgtatttcctccccattatttcaagcatggagatactgaattcaatctcgttgatggtgcaatcatcgctgatattccggccccggttgctctcagcgaggtgctccagcaagaaaaatacaagaataaagaaatccttttgctgtctataggaactggagttgtaaaacctggtgagggttattctgctaatcgtacttggactattttcgattggagtagtgaaactttaatcgggcttatgggtcacggaacgagagccatgtctgattattacgttggctcacatttcaaagcccttcaaccccagaataactacctccgaattcaggaatacgatttagatccggcactgggaagcattgatgatgcttcaacggaaaacatggagaatctggaaaaggtaggacagagtttgttgaacgaaccagttaaaaggatgaatctgaatacttttgtcgttgaagaaacaggtgaaggtaccaatgcagaagctttagacaggctggctcagattctttatgaagaaaagattactcgtggtctcggaaagatatctttggaagtggataacattgatccatatactgaacgtgttaggaaactgctattc

SEQ ID NO: 6 - protein, 390 aa, Artificial Sequence
  Description of Artificial Sequenceclone PIP-3 improved pentin lipid acyl hydrolase
MGSAFSTQAKASKDGNLVTVLAIDGGGIRGIIPGVILKQLEATLQRWDPSARLAEYFDVVAGTSTGGIIAAILTAPDPQNKDRPLYAAEEIIDFYIEHGPSIFNKSTACPLPGIFCPKYDGKYLQEIISQKLNETLLDQTTTNVVIPSFDIKLLRPTIFSTFKLEEVPELNVKLSDVCMGTSAAPIVFPPHYFKHGDTEFNLVDGAIIADIPAPVALSEVLQQEKYKNKEILLLSIGTGVVKPGEGYSANRTWTIFDWSSETLIGLMGHGTRAMSDYYVGSHFKALQPQNNYLRIQEYDLDPALGSIDDASTENMENLEKVGQSLLNEPVKRMNLNTFVVEETGEGTNAEALDRLAQILYEEKITRGLGKISLEVDNIDPYTERVRKLLF

SEQ ID NO: 7 - DNA, 1170 bp, Artificial Sequence
  Description of Artificial Sequenceclone PIP-5 improved pentin lipid acyl hydrolase
atgggatccgcattttccacacaagcgaaagcttctaaagatggaaacttagtcacagttcttgccattgatggaggtggtatcagaggaattatccccggagttattctcaaacaactagaagctactcttcagagatgggactcaagtgcaagactagcagagtattttgatgtggttgccgggacgagcactggagggattataactgccatcctgactgccccggacccacaaaacaaggaccgtcctttgtatgctgccgaagaaattatcgacttctacatagagcatggtccttccatttttaacaaatccaccgcctgctcgttgcctggtatctttcgtccaaagtatgatgggaagtatttacaagaaataataagccagaaattgaatgaaacactactagaccagacaacaacaaatgttgttatcccttccttcgacatcaagcttcttcgtccaaccatattctcaactttcaagttagaggaagttcctgagttaaatgtcaaactctccgatgtatgcatgggaacttcagcagcaccaatcgtatttcctccctattatttcaagcatggagatactgaattcaatctcgttgatggtgcaatcatcgctgatattccggccccggttgctctcagcgaggtgctccagcaagaaagatacaagaataaagaaatccttttgctgtctataggaactggagttgtaaaacctggtgagggttattctgctaatcgtacttggactattttcgatcggagtagtgaaactttaatcgggcttatgggacatggaacgagagccatgtctgattattacgttggctcacatttcaaagcccttcaaccccagaataactacctccgaattcaggaatacgatttagatccggcactggaaagcattgatgatgcttcaacggaaaacatggagaatctggaaaaggtaggacagagtttgttgaacgaaccagttaaaaggatgaatctgaatacttttgtcgttgaagaaacaggtgaaggtaccaatgcagaagctttagacaggctggctcagattctttatgaagaaaagattactcgtggtctcggaaagatatctttggaagtggataacattgatccatatactgaacgtgttaggaaactgctattc

SEQ ID NO: 8 - protein, 390 aa, Artificial Sequence
  Description of Artificial Sequenceclone PIP-5 improved pentin lipid acyl hydrolase
MGSAFSTQAKASKDGNLVTVLAIDGGGIRGIIPGVILKQLEATLQRWDSSARLAEYFDVVAGTSTGGIITAILTAPDPQNKDRPLYAAEEIIDFYIEHGPSIFNKSTACSLPGIFRPKYDGKYLQEIISQKLNETLLDQTTTNVVIPSFDIKLLRPTIFSTFKLEEVPELNVKLSDVCMGTSAAPIVFPPYYFKHGDTEFNLVDGAIIADIPAPVALSEVLQQERYKNKEILLLSIGTGVVKPGEGYSANRTWTIFDRSSETLIGLMGHGTRAMSDYYVGSHFKALQPQNNYLRIQEYDLDPALESIDDASTENMENLEKVGQSLLNEPVKRMNLNTFVVEETGEGTNAEALDRLAQILYEEKITRGLGKISLEVDNIDPYTERVRKLLF

SEQ ID NO: 9 - DNA, 1173 bp, Artificial Sequence
  Description of Artificial Sequenceclone PIP-6 improved pentin lipid acyl hydrolase
atgggatccgcattatccacacaagcgaaagcttctaaagatggaaacttagtcacagttcttgccattgatggaggtggtatcagaggaattatccccggagttattctcaaacaactagaagctactcttcagagatgggactcaagtgcaagactagcagagtattttgatgtggttgccgggacgagcactggagggattataactgccattctaactgccccggacccacaaaacaagggccgtcctttgtatgctgccgaagaaattatcaacttctacatagagcatggtccttccatttttaataaatccaccgcctgctcgttgcctggtatcttttgtccaaagtatgatgggaagtatttacaagaaataataagccagaaattgaatgaaacacgactagaccagacaacaacaaatgttgttatcccttccttcgacatcaagcttcttcgtccaaccatattctcaactttcaagttagaggaagttcctgagttaaatgtcaaactctccgatgtatgcatgggaacttcagcagcaccaatcgtatttcctccctattatttcaagcatggagatactgaattcaatctcgttgatggtgcaatcatcgctgatattccggccccggttgctctcagcgaggtgctccagcaagaaaaatacaagaataaagaaatccttttgctgtctataggaactggagttgtaaaacctggtgagggttattctgctaatcgtacttggactattttcgattggagtagtgaaactttaatcgggcttatgggtcatggaacgagagccatgtctgattattacgttggctcacatttcaaagcccttcaaccccagaataactacctccgaattcaggaatacgatttagatccggcactgggaagcattgatgatgcttcaacggaaaacatggagaatctggaaaaggtaggacagagtttgttgaacgaaccagttaaaaggatgaatctgaatacttttgtcgttgaagaaacaggtgaaggtaccaatgcagaagctttagacaggctggctcagattctttatgaagaaaagattactcgtggtctcggaaagatatctttggaagtggataacattgatccatatactgaacgtgttaggaaactgctattctga

SEQ ID NO: 10 - protein, 390 aa, Artificial Sequence
  Description of Artificial Sequenceclone PIP-6 improved pentin lipid acyl hydrolase
MGSALSTQAKASKDGNLVTVLAIDGGGIRGIIPGVILKQLEATLQRWDSSARLAEYFDVVAGTSTGGIITAILTAPDPQNKGRPLYAAEEIINFYIEHGPSIFNKSTACSLPGIFCPKYDGKYLQEIISQKLNETRLDQTTTNVVIPSFDIKLLRPTIFSTFKLEEVPELNVKLSDVCMGTSAAPIVFPPYYFKHGDTEFNLVDGAIIADIPAPVALSEVLQQEKYKNKEILLLSIGTGVVKPGEGYSANRTWTIFDWSSETLIGLMGHGTRAMSDYYVGSHFKALQPQNNYLRIQEYDLDPALGSIDDASTENMENLEKVGQSLLNEPVKRMNLNTFVVEETGEGTNAEALDRLAQILYEEKITRGLGKISLEVDNIDPYTERVRKLLF

SEQ ID NO: 11 - DNA, 1170 bp, Artificial Sequence
  Description of Artificial Sequenceclone PIP-8 improved pentin lipid acyl hydrolase
atgggatccgcattttccacccaagcgaaagcttctaaagatggaaacctagtcacagttcttgccattgatggaggtggtatcagaggaattatccccggagttattctcaaacaactagaagctactcttcagagatgggactcaagtgcaagactagcagagtattttgacgtggttgccgggacgagcactggagggattataactgccattctaactgccccggacccacaaaacaaggaccgtcctttgtatgctgccgaagaaattgtcggcttctacatagagcatggtccttccatttttaataaatccaccgcctgctcgttgcctggtatcttttgtccaaagtatgatgggaagtatttacaagaaataataagccagaaattgaatgaaacactactagaccagacaacaacaaatgttgttatcccttccttcgacatcaagcttcttcgtccaaccatattctcaactttcaagttagaggaagttcctgagttaaatgtcaaactctccgatgtatgcatgggaacttcagcagcaccaaccgtatttcctccctattatttcaagcatggagatactgaattcaatctcgttgatggtgcaatcatcgctggtattccggccccggttgctctcagcgaggtgctccagcaagaaaaatacaagaataaagaaatccttttgctgtctataggaactggagttgtaaaacctggtgagggttattctgctaatcgtacttggactattttcgattggagtagtgaaactttaatcgggcttatgggtcatggaacgagagccatgtctgattcttacgttggctcacatttcaaagcccttcaaccccagaataactacctccgaattcaggaatacgatttagatccggcactggaaagcattgatgatgcttcaacggaaaacatggagaatctggaaaaggtaggacagagtttgttgaacgaaccagttaaaaggatgaatctgaatacttttgtcgttgaagaaacaggtgaaggtaccaatgcagaagctttagacaggctggctcagattctttatgaagaaaagattactcgtggtctcggaaagatatctttggaagtggataacattgatccatatactgaacgtgttaggaaactgctattc

SEQ ID NO: 12 - protein, 390 aa, Artificial Sequence
  Description of Artificial Sequenceclone PIP-8 improved pentin lipid acyl hydrolase
MGSAFSTQAKASKDGNLVTVLAIDGGGIRGIIPGVILKQLEATLQRWDSSARLAEYFDVVAGTSTGGIITAILTAPDPQNKDRPLYAAEEIVGFYIEHGPSIFNKSTACSLPGIFCPKYDGKYLQEIISQKLNETLLDQTTTNVVIPSFDIKLLRPTIFSTFKLEEVPELNVKLSDVCMGTSAAPTVFPPYYFKHGDTEFNLVDGAIIAGIPAPVALSEVLQQEKYKNKEILLLSIGTGVVKPGEGYSANRTWTIFDWSSETLIGLMGHGTRAMSDSYVGSHFKALQPQNNYLRIQEYDLDPALESIDDASTENMENLEKVGQSLLNEPVKRMNLNTFVVEETGEGTNAEALDRLAQILYEEKITRGLGKISLEVDNIDPYTERVRKLLF

SEQ ID NO: 13 - DNA, 1173 bp, Artificial Sequence
  Description of Artificial Sequenceclone PIP-10 improved pentin lipid acyl hydrolase
atgggatccgcattttccacacaagcgaaagcttctaaagatggaaacttagtcacagttcttgccattgatggaggtggtatcagaggaattatccccggagttattctcaaacaactagaagctactcttcagagatgggactcgagtgcaagactagcagagtattttgatgtggttgccgggacgagcactggagggattataactgccattctaactgccccggacccacaaaacaaggaccgtcctctgtatgctgccggagaaattatcgacttctacatagagcatggtccttccatttttaataaatccaccgcctgctcgtcgcctggtatcttttgtccaaagtatgatgggaagtatttacaagaaataataagccagaaattgaatgaaacactactagaccagacaacaacaaatgttgttatcccttccttcgacatcaagcttcttcgtccaaccatattctcaactttcaagttagaggaagttcctgagttaaatgtcaaactctccgatgtatgcatgggaacttcagcagcaccaatcgtatttcctccctattatttcaagcatggagatactgaattcaatctcgttgatggtgcaatcatcgctgatattccggccccggttgctctcagcgaggtgctccagcaagaaaaatacaagaataaagaaatccttttgctgtctataggaactggagttgtaaaacctggtgagggttattctgctaatcgtacttggactattttcgattggagtagtgaaactttaatcgggcttatgggtcatggaacgagagccatgtctgattattacgttggctcacatttcaaagcccttcaaccccagagtaactacctccgaattcaggaatacgatttagatccggcactggaaagcattgatgatgcttcaacggaaaacatggagaatctggaaaaggtaggacagagtttgttgaacgaaccagttaaaaggatgaatctgaatacttttgtcgttgaagaaacaggtgaaggtaccaatgcagaagctttagacaggctggctcagattctttatgaagagaagattactcgtggtctcggaaagatatctttggaagtggataacattgatccatatactgaacgtgttaggaaactgctattctga

SEQ ID NO: 14 - protein, 390 aa, Artificial Sequence
  Description of Artificial Sequenceclone PIP-10 improved pentin lipid acyl hydrolase
MGSAFSTQAKASKDGNLVTVLAIDGGGIRGIIPGVILKQLEATLQRWDSSARLAEYFDVVAGTSTGGIITAILTAPDPQNKDRPLYAAGEIIDFYIEHGPSIFNKSTACSSPGIFCPKYDGKYLQEIISQKLNETLLDQTTTNVVIPSFDIKLLRPTIFSTFKLEEVPELNVKLSDVCMGTSAAPIVFPPYYFKHGDTEFNLVDGAIIADIPAPVALSEVLQQEKYKNKEILLLSIGTGVVKPGEGYSANRTWTIFDWSSETLIGLMGHGTRAMSDYYVGSHFKALQPQSNYLRIQEYDLDPALESIDDASTENMENLEKVGQSLLNEPVKRMNLNTFVVEETGEGTNAEALDRLAQILYEEKITRGLGKISLEVDNIDPYTERVRKLLF

SEQ ID NO: 15 - DNA, 1170 bp, Artificial Sequence
  Description of Artificial Sequenceclone PIP-11 improved pentin lipid acyl hydrolase
atgggatccgcattttccacacaagcgaaagcttctaaagatggaaacttagtcacagttcttgccattgatggaggtggtatcagaggaattatccccggagttattctcaaacaactagaagctactcttcagagatgggactcaagtgcaagactagcagagtattttgatgtggttgccgggacgagcactggagggattataactgccattctaactgccccggacccacaaaacaaggaccgtcctttgtatgctgccgaagaaattatcgacttctacatagagcatggtccttccatttttaataaatccaccgcctgctcgttgcctggtatcttttgtccaaagtatgatgggaagtatttacaagaaataataagccagaaattgaatgaaacactactggaccagacaacaacaaatgttgttatcccttccttcgacatcaagcttcttcgtccaaccatattctcaactttcaagttagaggaagttcctgagttaaatgtcaaactctccgatgtatgcatgggaacttcagcagcaccaatcgtatttcctccctattatttcaagcatggagatactgaattcaatctcgttgatggtgcaatcatcgctgatattccggccccggttgctctcagcgaggtgctccagcaagaaaaatacaagaataaagaaatccttttgctgtctataggaactggagttgtaaaacctggtgagggttattctgctaatcgtacttggactattttcgattggggtagtgaaactttaatcgggcctatgggtcacggaacgagagccatgtctgattattacgttggctcacatttcaaagcccttcaaccccagaataactacctccgaattcaggaatacgatttagatccggcactggaaagcattgatgatgcttcaacggaaaacatggagaatctggaaaaggtaggacagagtttgttgaacgaaccagttaaaaggatgaatctgaatacttttgtcgttgaagaaacaggtgaaggtaccaatgcagaagctttagacaggctggctcagattctttatgaagaaaagattactcgtggtctcggaaagatatctttggaagtggataacattgatccatatactgaacgcgttaggaaactgctattc

SEQ ID NO: 16 - protein, 390 aa, Artificial Sequence
  Description of Artificial Sequenceclone PIP-11 improved pentin lipid acyl hydrolase
MGSAFSTQAKASKDGNLVTVLAIDGGGIRGIIPGVILKQLEATLQRWDSSARLAEYFDVVAGTSTGGIITAILTAPDPQNKDRPLYAAEEIIDFYIEHGPSIFNKSTACSLPGIFCPKYDGKYLQEIISQKLNETLLDQTTTNVVIPSFDIKLLRPTIFSTFKLEEVPELNVKLSDVCMGTSAAPIVFPPYYFKHGDTEFNLVDGAIIADIPAPVALSEVLQQEKYKNKEILLLSIGTGVVKPGEGYSANRTWTIFDWGSETLIGPMGHGTRAMSDYYVGSHFKALQPQNNYLRIQEYDLDPALESIDDASTENMENLEKVGQSLLNEPVKRMNLNTFVVEETGEGTNAEALDRLAQILYEEKITRGLGKISLEVDNIDPYTERVRKLLF

SEQ ID NO: 17 - DNA, 1173 bp, Artificial Sequence
  Description of Artificial Sequenceclone PIP-12 improved pentin lipid acyl hydrolase
atgggatccgcattttccacacaagcgaaagcttctaaagatggaaacttagtcacagttcttgccattgatggaggtggtatcagaggaattatccccggagttattctcaaacaactagaagctactcttcagagatgggacccaagtgcaagactagcagagtattttgatgtggttgccgggacgagcactggagggattataactgccattctaactgccccggacccacaaaacaaggaccgtcctttgtatgctgccgaagaaattatcgacttctacatagagcatggtccttccatttttaataaatccaccgcctgctcgttgcctggtatcttttgtccaaagtatgatgggaagtatttacaagaaataataagccagaaattgaatgaaacactactagaccagacaacaacaaatgttgttatcccttccttcgacatcaagcttcttcgtccaaccatattctcaactttcaagttagaggaagttcctgagttaaatgtcaaactctccgatgtatgcatgggaacttcagcagcaccaatcgtatttcctccctattatttcaagcatggagatactgaattcaatctcgttgatggtgcaatcatcgctgatactccggccccggttgctctcagcgaggtgctccagcaagaaaaatacaagaataaagaaatccttttgctgtctataggaactggagttgtaaaacctggtgagggttattctgctaatcgtacttggactattttcgattggagtagtgaaactttaatcgggcttatgggtcatggaacgagagccatgtctgattattacgttggctcacatttcaaagcccttcaaccccagaataactacctccgaattcaggaatacgatttagatccagcgctggaaagcattgatgatgcttcaacggaaaacatggagaatctggaaaaggtaggacagagtttgttgaacgaaccagttaaaaggatgaatctgaatacttttgtcgttgaagaaacaggtgaaggtaccaatgcagaagctttagacaggctggctcagattctttatgaagaaaagattactcgtggtctcggaaagatatctttggaagtgggtaacattgatccatatactgaacgtgttaggaaactgctattctga

SEQ ID NO: 18 - protein, 390 aa, Artificial Sequence
  Description of Artificial Sequenceclone PIP-12 improved pentin lipid acyl hydrolase
MGSAFSTQAKASKDGNLVTVLAIDGGGIRGIIPGVILKQLEATLQRWDPSARLAEYFDVVAGTSTGGIITAILTAPDPQNKDRPLYAAEEIIDFYIEHGPSIFNKSTACSLPGIFCPKYDGKYLQEIISQKLNETLLDQTTTNVVIPSFDIKLLRPTIFSTFKLEEVPELNVKLSDVCMGTSAAPIVFPPYYFKHGDTEFNLVDGAIIADTPAPVALSEVLQQEKYKNKEILLLSIGTGVVKPGEGYSANRTWTIFDWSSETLIGLMGHGTRAMSDYYVGSHFKALQPQNNYLRIQEYDLDPALESIDDASTENMENLEKVGQSLLNEPVKRMNLNTFVVEETGEGTNAEALDRLAQILYEEKITRGLGKISLEVGNIDPYTERVRKLLF

SEQ ID NO: 19 - DNA, 1170 bp, Artificial Sequence
  Description of Artificial Sequenceclone PIP-15 improved pentin lipid acyl hydrolase
atgggatccgcattatccacacaagcgaaagcttctaaagatggaaacttagtcacagttcttgccattgatggaggtggtatcagaggaatcatccccggagttattctcaaacaactagaagctactcttcagagatgggactcaagtgcaagactagcagagtattttgatgtggttgccgggacgagcactggagggattataactgccattctaactgccccggacccacaaaacaaggaccgtcctttgtatgctgccgaagaaattatcgacttctacatagagcatggtccttccatttttaataaatccaccgcctgctcgttgcctggtatcttttgtccaaagtatgatgggaagtatttacaagaaataataagccagaaattgaatgaaacactactagaccagacaacaacaaatgttgttatcccttccttcgacatcaagcttcttcgtccaaccatattctcaactttcaagttagaggaagttcctgagttaaatgtcaaactctccgatgtatgcatgggaacttcagcagcgccaatcgtatttcctccctattatttcaagcatggagatactgaattcaatctcgttgatggtgcaatcatcgctgatattccggccccggttgctctcagcgaggtgctccagcaagaaaaatacaagaataaagaaatccttttgctgtctataggaactggagttgtaaaacctggtgagggttattctgctaatcgtacttggactattttcgattggagtagtgaaactttaatcgggcttatgggtcatggaacgagagccatgtctgattattacgttggctcacatttcaaagcccttcaaccccagaataactacctccgaattcaggaatacgatttagatccggcactggaaagcattgatgatgcttcaacggaaaacatggagaatctggaaaaggtaggacagagtttgttgaacgaaccagttaaaaggatgaatctgaatacttttgtcgttgaagaaacaggtgaaggtaccaatgcagaagctttagacaggctggctcggattctttatgaagaaaagattactcgtggtctcggaaagatatctttggaagtggataacattgatccatatactgaacgtgttaggaaactgctattc

SEQ ID NO: 20 - protein, 390 aa, Artificial Sequence
  Description of Artificial Sequenceclone PIP-15 improved pentin lipid acyl hydrolase
MGSALSTQAKASKDGNLVTVLAIDGGGIRGIIPGVILKQLEATLQRWDSSARLAEYFDVVAGTSTGGIITAILTAPDPQNKDRPLYAAEEIIDFYIEHGPSIFNKSTACSLPGIFCPKYDGKYLQEIISQKLNETLLDQTTTNVVIPSFDIKLLRPTIFSTFKLEEVPELNVKLSDVCMGTSAAPIVFPPYYFKHGDTEFNLVDGAIIADIPAPVALSEVLQQEKYKNKEILLLSIGTGVVKPGEGYSANRTWTIFDWSSETLIGLMGHGTRAMSDYYVGSHFKALQPQNNYLRIQEYDLDPALESIDDASTENMENLEKVGQSLLNEPVKRMNLNTFVVEETGEGTNAEALDRLARILYEEKITRGLGKISLEVDNIDPYTERVRKLLF

SEQ ID NO: 21 - DNA, 1170 bp, Artificial Sequence
  Description of Artificial Sequenceclone PIP-16 improved pentin lipid acyl hydrolase
atgggatccgcattttccacacaagcgaaatcttctaaagatggaaacttagtcacagttcttgccattgacggaggtggtatcagaggaattatccccggagttattctcaaacaactagaagctactcttcagagatgggactcaagtgcaagactagcagagtattttgatgtggttgccgggacgagcactggagggattataactgccattctaactgccccggacccacaaaacaaggaccgtcctttgtatgctgccgaagaaattatcgacttctacatagagcatggtccttccatttttaataaatccaccgcctgctcgttgcctggtatcttttgtccaaagtatgatgggaagtatttacaagaaataataagccagaaattgaatgaaacactactagaccagacaacaacaaatgttgttatcccttccttcgacatcaagcttcttcgtccaaccatattctcaactttcaagttagaggaagttcctgagttaaatgtcaaactctccgatgtatgcatgggaacttcagcagcaccaatcgtatttcctccctattatttcaagcatggagatactgaattcaatctcgttgatggtgcaatcatcgctggtattccggccccggttgctctcagcgaggtgctccagcaagaaaaatacaagaataaagaaatccttttgctgtctataggaactggagttgtaaaacctggtgagggttattctgctaatcgtacttggactattttcgattggagtagtgaaactttaatcgggcttatgggtcatggaacgagagccatgtctgattattacgttggctcacatttcaaagcccttcaaccccagaataactacctccgaattcaggaatacgatttagatccggcactggaaagcattgatgatgcttcaacggaaaacatggagaatctggaaaaggtaggacagagtttgttgaacgaaccagttaaaaggatgaatctgaatacttttgtcgttgaagaaacaggtgaaggtaccaatgcagaagctttagacaggctggctcagattctttatgaagaaaagattactcgtggtctcggaaagatatctttggaagtggataacattgatccatatactgaacgtgttaggaaactgctattc

SEQ ID NO: 22 - protein, 390 aa, Artificial Sequence
  Description of Artificial Sequenceclone PIP-16 improved pentin lipid acyl hydrolase
MGSAFSTQAKSSKDGNLVTVLAIDGGGIRGIIPGVILKQLEATLQRWDSSARLAEYFDVVAGTSTGGIITAILTAPDPQNKDRPLYAAEEIIDFYIEHGPSIFNKSTACSLPGIFCPKYDGKYLQEIISQKLNETLLDQTTTNVVIPSFDIKLLRPTIFSTFKLEEVPELNVKLSDVCMGTSAAPIVFPPYYFKHGDTEFNLVDGAIIAGIPAPVALSEVLQQEKYKNKEILLLSIGTGVVKPGEGYSANRTWTIFDWSSETLIGLMGHGTRAMSDYYVGSHFKALQPQNNYLRIQEYDLDPALESIDDASTENMENLEKVGQSLLNEPVKRMNLNTFVVEETGEGTNAEALDRLAQILYEEKITRGLGKISLEVDNIDPYTERVRKLLF

SEQ ID NO: 23 - DNA, 1170 bp, Artificial Sequence
  Description of Artificial Sequenceclone PIP-20 improved pentin lipid acyl hydrolase
atgggatccgcattttccacacaagcgaaagcttctaaagatggaaacttagtcacagttcttgccattgatggaggtggtatcagaggaattatccccggagttatcctcaaacaactagaagctactcttcagagatgggacccaagtgcaagactagcagagtattttgatgtggttgccgggacgagcactggagggattataactgccattctaactgccccggacccacaaaacaaggaccgtcctttgtatgctgccgaagaaattatcgacttctacgtagagcatggtccttccatttttaataaatccaccgcctgctcgttgcctggtatcttttgtccaaagtatgatgggaagtatttacaagaaataataagccagaaattgaatgaaacactactagaccagacaacaacaaatgttgttatcccttccttcgacatcaagcttcttcgtccaaccatattctcaactttcaagttagaggaagttcctgagttaaatgtcaaactctccgatgtatgcatgggaacttcagcagcaccaatcgtatttcctccctattatttcaagcatggagatactgaattcaatctcgttgatggtgcaatcatcgctgatattccggccccggttgctctcagcgaggtgctccagcaagaaaaatacaagaataaagaaatccttttgctgtctataggaactggagttgtaaaacctggtgagggttgttctgctaatcgtacttggactattttcgattggagtagtgaaactttaatcgggcttatgggtcatggaacgagagccatgtctgattattacgttggctcacatttcaaagcccttcaaccccagaataactacctccgaattcaggaatacgatttagatccggcactgggaggcattgatgatgcttcaacggaaaacatggagaatctggaaaaggtaggacagagtttgttgaacgaaccagttaaaaggatgaatctgaatacttttgtcgttgaagaaacaggtgaaggtaccaatgcagaagctttagacaggctggctcagattctttatgaagaaaagattactcgtggtctcggaaagatatctttggaagtggataacattgatccatatactgaacgtgttaggaaactcctattc

SEQ ID NO: 24 - protein, 390 aa, Artificial Sequence
  Description of Artificial Sequenceclone PIP-20 improved pentin lipid acyl hydrolase
MGSAFSTQAKASKDGNLVTVLAIDGGGIRGIIPGVILKQLEATLQRWDPSARLAEYFDVVAGTSTGGIITAILTAPDPQNKDRPLYAAEEIIDFYVEHGPSIFNKSTACSLPGIFCPKYDGKYLQEIISQKLNETLLDQTTTNVVIPSFDIKLLRPTIFSTFKLEEVPELNVKLSDVCMGTSAAPIVFPPYYFKHGDTEFNLVDGAIIADIPAPVALSEVLQQEKYKNKEILLLSIGTGVVKPGEGCSANRTWTIFDWSSETLIGLMGHGTRAMSDYYVGSHFKALQPQNNYLRIQEYDLDPALGGIDDASTENMENLEKVGQSLLNEPVKRMNLNTFVVEETGEGTNAEALDRLAQILYEEKITRGLGKISLEVDNIDPYTERVRKLLF

SEQ ID NO: 25 - DNA, 1170 bp, Artificial Sequence
  Description of Artificial Sequenceclone PIP-53 improved pentin lipid acyl hydrolase
atgggatccgcattttccacacaagcgaaagcttctaaagatggaaacttagtcacagttcttgccattgatggaggtggtatcagaggaattatccccggagttattctcaaacaactagaagctactcttcagagatgggactcaagtgcaagactagcagagtattttgatgtggttgccgggacgagcactggagggattataactgccattctaactgccccggacccacaaaacaaggaccgtcctttgtatgctgccgaagaaattatcgacttctacatagagcatggtccttccatttttaataaatccaccgcctgctcgttgcctggtatcttttgtccaaagtatgatgggaagtatttacaagaaataataagccagaaattgaatgaaacactactagaccagacaacaacaaatgttgttatcccttccttcgacatcaagcttcttcgtccaaccatattctcaactttcaagttagaggaagttcctgagttaaatgtcaaactctccgatgtatgcatgggaacttcagcagcaccaatcgtatttcctccctattatttcaagcatggggatactgaattcaatctcgttgatggtgcaatcatcgctggtattccggccccggttgctctcagcgaggtgctccagcaagaaaaatacaagaataaagaaatccttttgctgtctataggaactggagttgtaaaacctggtgagggttattctgctaaccgtacttggactattttcgattggagtagtgaaactttaatcgggcttatgggtcatggaacgagagccatgtctgattattacgttggctcacatttcaaagcccttcaaccccagaataactacctccgaattcaggaatacgacttagatccggcactggaaagcattgatgatgcttcaacggaaaacatggagaatctggaaaaggtaggacagagtttgttgaacgaaccagttaaaaggatgaatctgaatacttttgtcgttgaagaaacaggtgaaggtaccaatgcagaagctttagacaggctggctcagattctttatgaagaaaagattactcgtggtctcggaaagatatctttggaagtggataacattgatccatatactgaacgtgttaggaaactgctattc

SEQ ID NO: 26 - protein, 390 aa, Artificial Sequence
  Description of Artificial Sequenceclone PIP-53 improved pentin lipid acyl hydrolase
MGSAFSTQAKASKDGNLVTVLAIDGGGIRGIIPGVILKQLEATLQRWDSSARLAEYFDVVAGTSTGGIITAILTAPDPQNKDRPLYAAEEIIDFYIEHGPSIFNKSTACSLPGIFCPKYDGKYLQEIISQKLNETLLDQTTTNVVIPSFDIKLLRPTIFSTFKLEEVPELNVKLSDVCMGTSAAPIVFPPYYFKHGDTEFNLVDGAIIAGIPAPVALSEVLQQEKYKNKEILLLSIGTGVVKPGEGYSANRTWTIFDWSSETLIGLMGHGTRAMSDYYVGSHFKALQPQNNYLRIQEYDLDPALESIDDASTENMENLEKVGQSLLNEPVKRMNLNTFVVEETGEGTNAEALDRLAQILYEEKITRGLGKISLEVDNIDPYTERVRKLLF

SEQ ID NO: 27 - DNA, 1170 bp, Artificial Sequence
  Description of Artificial Sequenceclone PIP-55 improved pentin lipid acyl hydrolase
atgggatccgcattttccacacaagcgaaagcttctaaagatggaaacttagtcacagttcttgccattgatggaggtggtatcagaggaattatccccggagttattctcaaacaactagaagctactcttcagagatgggacccaagtgcaagactagcagagtattttgatgtggttgccgggacgagcactggagggattataactgccattctaactgccccggacccacaaaacaaggaccgtcctttgtatgctgccgaagaaattatcgacttctacatagagcatggtccttccatttttaataaatccaccgcctgctcgttgcctggtatcttttgtccaaagtatgatgggaagtatttacaagaaataataagccagaaattgaatgaaacactactagaccagacaacaacaaatgttgttatcccttccttcgacatcaagcttcttcgtccaaccatattctcaactttcaagttagaggaagttcctgagttaaatgtcaaactctccgatgtatgcatgggaacttcagcagcaccaatcgtatttcctccctattatttcaagcatggagatactgaattcaatctcgttgatggtgcaatcatcgctggtattccggccccggttgctctcagcgaggtgctccggcaagaaaaatacaagaataaagaaatccttttgctgtctataggaactggagttgtaaaacctggtgagggttattctgctaatcgtacttggactattttcgattggagtagtgaaactttaatcgggcttatgggtcatggaacgagagccatgtctgattattacgttggctcacttttcaaagcccttcaaccccagaataactacctccgaattcaggaatacgatttagatccggcactggaaagcattgatgatgcttcaacggaaaacatggggaatctggaaaaggtaggacagagtttgttgaacgaaccagttaaaaggatgaatctgaatacttttgtcgttgaagaaacaggtgaaggtaccaatgcagaagctttagacaggctggctcagattctttatgaagaaaagattactcgtggtctcggaaagatatctttggaagtggataacattgatccatatactgaacgtgtcaggaaactgctattc

SEQ ID NO: 28 - protein, 390 aa, Artificial Sequence
  Description of Artificial Sequenceclone PIP-55 improved pentin lipid acyl hydrolase
MGSAFSTQAKASKDGNLVTVLAIDGGGIRGIIPGVILKQLEATLQRWDPSARLAEYFDVVAGTSTGGIITAILTAPDPQNKDRPLYAAEEIIDFYIEHGPSIFNKSTACSLPGIFCPKYDGKYLQEIISQKLNETLLDQTTTNVVIPSFDIKLLRPTIFSTFKLEEVPELNVKLSDVCMGTSAAPIVFPPYYFKHGDTEFNLVDGAIIAGIPAPVALSEVLRQEKYKNKEILLLSIGTGVVKPGEGYSANRTWTIFDWSSETLIGLMGHGTRAMSDYYVGSLFKALQPQNNYLRIQEYDLDPALESIDDASTENMGNLEKVGQSLLNEPVKRMNLNTFVVEETGEGTNAEALDRLAQILYEEKITRGLGKISLEVDNIDPYTERVRKLLF

SEQ ID NO: 29 - DNA, 1170 bp, Artificial Sequence
  Description of Artificial Sequenceclone PIP-56 improved pentin lipid acyl hydrolase
atgggatccgcattttccacacaagcgaaagcttctaaagatggaaacttagtcacagttcttgccattgatggaggtggtatcagaggaattatccccggagttattctcaaacaactagaagctactcttcagagatgggactcaagtgcaagactagcagagtattttgatgtggttgccgggacgagcactggagggattataactgccattctaactgccccggacccacaaaacaaggaccgtcctttgtatgctgccgaagaaattatcgacttctacatagagcatggtccttccatttttaataaatccaccgcctgctcgttgcctggtatcttttgtccaaagtatgatgggaagtatttacaagaaataataagccagaaattgaatgaaacactactagaccagacaacaacaaatgttgttatcccttccttcgacatcaagcttcttcgtccaaccatattctcaactttcaagttagaggaagttcctgagttaaatgtcaaactctccgatgtatgcatgggaacttcagcagcaccaatcgtatttcctccctattatttcaagcatggagatactgaattcaatctcgttgatggtgcaatcatcgctggtattccggccccggttgctctcagcgaggtgctccagcaagaaaaatacaagaataaagaaatccttttgctgtctataggaactggagttgtaaaacctggtgagggttattctgctaatcgtacttggactattttcgattggagtagtgaaactttaatcgggcttatgggtcatggaacgagagccatgtctgattattacgttggctcacatttcaaagcccttcaaccccagaataactacctccgaattcaggaatacgatttagatccggcactggaaagcattgatgatgcctcaacggaaaacatggagaatctggaaaaggtaggacagagtttgttgaacgaaccagttaaaaggatgaatctgaatacttttgtcgttgaagaaacaggtgaaggtaccaatgcagaagctttagacaggctggctcagattctttatgaagaaaagattactcgtggtctcggaaagatatctttggaagtggataacattgatccatatactgaacgtgttaggaaactgctattc

SEQ ID NO: 30 - protein, 390 aa, Artificial Sequence; identical to SEQ ID NO: 28
  Description of Artificial Sequenceclone PIP-56 improved pentin lipid acyl hydrolase

SEQ ID NO: 31 - DNA, 1170 bp, Artificial Sequence
  Description of Artificial Sequenceclone PIP-58 improved pentin lipid acyl hydrolase
atgggatccgcattttccacacaagcgaaagcctctaaagatggaaacttagtcacagttcttgccattgatggaggtggtatcagaggaattatccccggagttattctcaaacaactagaagctactcttcagagatgggactcaagtgcaagactagcagagtattttgatgtggttgccgggacgagcactggagggattataactgccattctaactgccccggacccacaaaacaaggaccgtcctttgtatgctgccgaagaaattatcgacttctacatagagcatggtccttccatttttaataaatccaccgcctgctcgttgcctggtatcttttgtccaaagtatgatgggaagtatttacaagaaataataagccagaaattgaatgaaacactactagaccagacaacaacaaatgttgttatcccttccttcgacatcaagcttcttcgtccaaccatattctcaactttcaagttagaggaagttcctgagttaaatgtcaaactctccgatgtatgcatgggaacttcagcagcaccaatcgtatttcctccctattatttcaagcatggagatactgaattcaatctcgttgatggtgcaatcatcgctgatactccggccccggttgctctcagcgaggtgctccagcaagaaaaatacaagaataaagaaatccttttgctgtctataggaactggagttgtaaaacctggtgagggttattctgctaatcgtacttggactattttcgattggagtagtgaaactttaatcgggcttatgggtcatggaacgagagccatgtctgattattacgttggctcacatttcaaagcccttcaaccccagaataactacctccgaattcaggaatacgatttagatccggcactggaaagcattgatgatgcttcaacggaaaacatggagaatctggaaaaggtaggacagagtttgttgaacgaaccagttaaaaggatgaatctgaatacttttgtcgttgaagaaacaggtgaaggtaccaatgcagaagctttagacaggctggctcagattctttatgaggaaaagattactcgtggtctcggaaagatatctttggaagtggataacattgatccatatactgaacgtgttaggaaactgctattc

SEQ ID NO: 32 - protein, 390 aa, Artificial Sequence
  Description of Artificial Sequenceclone PIP-32 improved pentin lipid acyl hydrolase
MGSAFSTQAKASKDGNLVTVLAIDGGGIRGIIPGVILKQLEATLQRWDSSARLAEYFDVVAGTSTGGIITAILTAPDPQNKDRPLYAAEEIIDFYIEHGPSIFNKSTACSLPGIFCPKYDGKYLQEIISQKLNETLLDQTTTNVVIPSFDIKLLRPTIFSTFKLEEVPELNVKLSDVCMGTSAAPIVFPPYYFKHGDTEFNLVDGAIIADTPAPVALSEVLQQEKYKNKEILLLSIGTGVVKPGEGYSANRTWTIFDWSSETLIGLMGHGTRAMSDYYVGSHFKALQPQNNYLRIQEYDLDPALESIDDASTENMENLEKVGQSLLNEPVKRMNLNTFVVEETGEGTNAEALDRLAQILYEEKITRGLGKISLEVDNIDPYTERVRKLLF

SEQ ID NO: 33 - DNA, 1170 bp, Artificial Sequence; identical to SEQ ID NO: 31
  Description of Artificial Sequenceclone PIP-62 improved pentin lipid acyl hydrolase

SEQ ID NO: 34 - protein, 390 aa, Artificial Sequence; identical to SEQ ID NO: 32
  Description of Artificial Sequenceclone PIP-62 improved pentin lipid acyl hydrolase

SEQ ID NO: 35 - DNA, 1170 bp, Artificial Sequence
  Description of Artificial Sequenceclone PIP-63 improved pentin lipid acyl hydrolase
atgggatccgcattttccacacaagcgaaagcttctaaagatggaaactttgtcacagttcttgccgttgatggaggtggtatcagaggaattatccccggagttattctcaaacaactagaagctactcttcagagatgggactcaagtgcaagactagcagagtattttgatgtggttgccgggacgagcaccggagggattataactgccattctaactgccccggacccacaaaacaaggaccgtcctttgtatgctgccgaagaaattatcgacttctacatagagcatggtccttccatttttaataaatccaccgcctgctcgttgcctggtatcttttgtccaaagtatgatgggaagtatttacaagaaataataagccagaaattgaatgaaacactactagaccagacaacaacaaatgttgttatcccttccttcgacatcaagcttcttcgtccaaccatattctcaactttcaagttagaggaagttcctgagttaaatgtcaaactctccgatgtatgcacgggaacttcagcagcaccaatcgtatttcctccctattatttcaagcatggagatactgaattcaatctcgttgatggtgcaatcatcgctgataatccggccccggttgccctcagcgaggtgctccagcaagaaaaatacaagaataaagaaatccttttgctgtctataggaactggagttgtaaaacctggtgagggttattctgctaatcgtacttggactattttcgattggagtagtgaaactttaatcgggcttatgggtcatggaacgagagccatgtctgattattacgttggctcacatttcaaagcccttcaaccccggaataactacctccgaattcaggaatacgatttagatccggcactggaaagcattgatgatgcttcaacggaaaacatggagaatctggaaaaggtaggacagagtttgttgaacgaaccagttaaaaggatgaatctgaatacttttgtcgttgaagaaacaggtgaaggtaccaatgcagaagctttagacaggctggctcagattctttatgaagaaaagattactcgtggtctcggaaagatatctttggaagtggataacattgatccatatactgaacgtgttaggaaactgctattc

SEQ ID NO: 36 - protein, 390 aa, Artificial Sequence
  Description of Artificial Sequenceclone PIP-63 improved pentin lipid acyl hydrolase
MGSAFSTQAKASKDGNFVTVLAVDGGGIRGIIPGVILKQLEATLQRWDSSARLAEYFDVVAGTSTGGIITAILTAPDPQNKDRPLYAAEEIIDFYIEHGPSIFNKSTACSLPGIFCPKYDGKYLQEIISQKLNETLLDQTTTNVVIPSFDIKLLRPTIFSTFKLEEVPELNVKLSDVCTGTSAAPIVFPPYYFKHGDTEFNLVDGAIIADNPAPVALSEVLQQEKYKNKEILLLSIGTGVVKPGEGYSANRTWTIFDWSSETLIGLMGHGTRAMSDYYVGSHFKALQPRNNYLRIQEYDLDPALESIDDASTENMENLEKVGQSLLNEPVKRMNLNTFVVEETGEGTNAEALDRLAQILYEEKITRGLGKISLEVDNIDPYTERVRKLLF

SEQ ID NO: 37 - DNA, 1170 bp, Artificial Sequence
  Description of Artificial Sequenceclone PIP-64 improved pentin lipid acyl hydrolase
atgggatccgcattttccacacaagcgaaagcttctaaagatggaaacttagtcacagttcttgccattgatggaggtggtatcagaggaattatccccggagttattctcaaacaactagaagctactcttcagagatgggactcaagtgcaagactagcagagtattttgatgtggttgccgggacgagcactggagggattataactgccatcctaactgccccggacccacaaaacaaggaccgtcctttgtatgctgccgaagaaattatcgacttctacatagagcatggtccttccatttttaataaatccaccgcctgctcgttgcctggtatcttttgtccaaagtatgatgggaagtatttacaagaaataataagccagaaattgaatgaaacactactagaccagacaacaacaaatgttgttatcccttccttcgacatcaagcttcttcgtccaaccatattctcaactttcaagttagaggaagttcctgagttaaatgtcaaactctccgatgtatgcatgggaacttcagcagcaccagtcgtatttcctccctattatttcaagcatggagatactgaattcaatctcgttgatggtgcaaccatcgctgataatccggccccggttgctctcagcgaggtgctccagcaagaaaaatacaagaataaagaaatccttttgctgtctataggaactggagttgtaaaacctggtgagggttattctgctaatcgtacttggactattttcgattggagtagtgaaactttaatcgggcttatgggtcatggaacgagagccatgtctgattattacgttggctcacatttcaaagcccttcaaccccagaataactacctccgaattcaggaatacgatttagatccggcactggaaagcattgatgatgcttcaacggaaaacatggagaatctggaaaaggtaggacagagtttgttgaacgaaccagttaaaaggatgaatctgaatacttttgtcgttgaagaaacaggtgaaggtaccaatgcagaagctttagacaggctggctcagattctttatgaagaaaagattactcgtggtctcggaaagatatctttggaagtggataacattgatccatatactgaacgtgttaggaaactgctattc

SEQ ID NO: 38 - protein, 390 aa, Artificial Sequence
  Description of Artificial Sequenceclone PIP-64 improved pentin lipid acyl hydrolase
MGSAFSTQAKASKDGNLVTVLAIDGGGIRGIIPGVILKQLEATLQRWDSSARLAEYFDVVAGTSTGGIITAILTAPDPQNKDRPLYAAEEIIDFYIEHGPSIFNKSTACSLPGIFCPKYDGKYLQEIISQKLNETLLDQTTTNVVIPSFDIKLLRPTIFSTFKLEEVPELNVKLSDVCMGTSAAPVVFPPYYFKHGDTEFNLVDGATIADNPAPVALSEVLQQEKYKNKEILLLSIGTGVVKPGEGYSANRTWTIFDWSSETLIGLMGHGTRAMSDYYVGSHFKALQPQNNYLRIQEYDLDPALESIDDASTENMENLEKVGQSLLNEPVKRMNLNTFVVEETGEGTNAEALDRLAQILYEEKITRGLGKISLEVDNIDPYTERVRKLLF

SEQ ID NO: 39 - DNA, 1170 bp, Artificial Sequence
  Description of Artificial Sequenceclone PIP-67 improved pentin lipid acyl hydrolase
atgggatccgcattttccacacaagcgaaagcttctaaagatggaaacttagtcacagttcttgccattgatggaggtggtatcagaggaattatccccggagttattctcaaacaactagaagctactcttcagagatgggactcaagtgcaagactagcagagtattttgatgtggttgccgggacgagcactggagggattataaccgccattctaactgccccggacccacaaaacaaggaccgtcctttgtatgctgccgaagaaattatcgacttctacatagagcatggtccttccatttttaataaatccaccgcctgctcgttgcctggtatcttttgtccaaagtatgatgggaagtatttacaagaaataataagccagaaattgaatgaaacactactagaccagacaacaacaaatgttgttatcccttccttcgacatcaagcttcttcgtccaaccatattctcaactttcaagttagaggaagttcctgagttaaatgtcaaactctccgatgtatgcatgggaacttcagcagcaccaatcgtatttcctccctattatttcaagcatggagatactgaattcaacctcgttgatggtgcaatcatcgctggtattccggccccggttgctctcagcgaggtgctccagcaagaaaaatacaagaataaagaaatccttttgctgtctataggaactggagttgtaaaacctggtgagggttattctgctaatcgtacttggactattttcgattggagtagtgaaactttaatcgggcttatgggtcatggaacgagagccatgtctgattattacgttggctcacatttcaaagcccttcaaccccagaataactacctccgaattcaggaatacgatttagatccggcactggaaagcattgatgatgcttcaacggaaaacatggagaatctggaaaaggtaggacagagtttgttgaacgaaccagttaaaaggatgaatctgaatacttttgccgttgaagaaacaggtgaaggtaccaatgcagaagctttagacaggctggctcagattctttatgaagaaaagattactcgtggtctcggaaagatatctttggaagtggataacattgatccatatactgaacgtgttaggaaactgctattc

SEQ ID NO: 40 - protein, 390 aa, Artificial Sequence
  Description of Artificial Sequenceclone PIP-67 improved pentin lipid acyl hydrolase
MGSAFSTQAKASKDGNLVTVLAIDGGGIRGIIPGVILKQLEATLQRWDSSARLAEYFDVVAGTSTGGIITAILTAPDPQNKDRPLYAAEEIIDFYIEHGPSIFNKSTACSLPGIFCPKYDGKYLQEIISQKLNETLLDQTTTNVVIPSFDIKLLRPTIFSTFKLEEVPELNVKLSDVCMGTSAAPIVFPPYYFKHGDTEFNLVDGAIIAGIPAPVALSEVLQQEKYKNKEILLLSIGTGVVKPGEGYSANRTWTIFDWSSETLIGLMGHGTRAMSDYYVGSHFKALQPQNNYLRIQEYDLDPALESIDDASTENMENLEKVGQSLLNEPVKRMNLNTFAVEETGEGTNAEALDRLAQILYEEKITRGLGKISLEVDNIDPYTERVRKLLF

SEQ ID NO: 41 - protein, 390 aa, Artificial Sequence
  Description of Artificial Sequencemodified native pentin lipid acyl hydrolase
MGSAFSTQAKASKDGNLVTVLAIDGGGIRGIIPGVILKQLEATLQRWDSSARLAEYFDVVAGTSTGGIITAILTAPDPQNKDRPLYAAEEIIDFYIEHGPSIFNKSTACSLPGIFCPKYDGKYLQEIISQKLNETLLDQTTTNVVIPSFDIKLLRPTIFSTFKLEEVPELNVKLSDVCMGTSAAPIVFPPYYFKHGDTEFNLVDGAIIADIPAPVALSEVLQQEKYKNKEILLLSIGTGVVKPGEGYSANRTWTIFDWSSETLIGLMGHGTRAMSDYYVGSHFKALQPQNNYLRIQEYDLDPALESIDDASTENMENLEKVGQSLLNEPVKRMNLNTFVVEETGEGTNAEALDRLAQILYEEKITRGLGKISLEVDNIDPYTERVRKLLF

SEQ ID NO: 42 - protein, 387 aa, Artificial Sequence
  Description of Artificial Sequencetruncated native wild-type pentin lipid acyl hydrolase
AFSTQAKASKDGNLVTVLAIDGGGIRGIIPGVILKQLEATLQRWDSSARLAEYFDVVAGTSTGGIITAILTAPDPQNKDRPLYAAEEIIDFYIEHGPSIFNKSTACSLPGIFCPKYDGKYLQEIISQKLNETLLDQTTTNVVIPSFDIKLLRPTIFSTFKLEEVPELNVKLSDVCMGTSAAPIVFPPYYFKHGDTEFNLVDGAIIADIPAPVALSEVLQQEKYKNKEILLLSIGTGVVKPGEGYSANRTWTIFDWSSETLIGLMGHGTRAMSDYYVGSHFKALQPQNNYLRIQEYDLDPALESIDDASTENMENLEKVGQSLLNEPVKRMNLNTFVVEETGEGTNAEALDRLAQILYEEKITRGLGKISLEVDNIDPYTERVRKLLF

SEQ ID NO: 43 - protein, 407 aa, Pentaclethra macroloba
  wild-type pentin lipid acyl hydrolase
MKSKMAMLLLLFCVLSNQLVAFSTQAKASKDGNLVTVLAIDGGGIRGIIPGVILKQLEATLQRWDSSARLAEYFDVVAGTSTGGIITAILTAPDPQNKDRPLYAAEEIIDFYIEHGPSIFNKSTACSLPGIFCPKYDGKYLQEIISQKLNETLLDQTTTNVVIPSFDIKLLRPTIFSTFKLEEVPELNVKLSDVCMGTSAAPIVFPPYYFKHGDTEFNLVDGAIIADIPAPVALSEVLQQEKYKNKEILLLSIGTGVVKPGEGYSANRTWTIFDWSSETLIGLMGHGTRAMSDYYVGSHFKALQPQNNYLRIQEYDLDPALESIDDASTENMENLEKVGQSLLNEPVKRMNLNTFVVEETGEGTNAEALDRLAQILYEEKITRGLGKISLEVDNIDPYTERVRKLLF

SEQ ID NO: 44 - DNA, 45 bp, Artificial Sequence
  Description of Artificial Sequenceoligo BC24
ggattatacacatatgggattcgcattttccacacaagcgaaagc

SEQ ID NO: 45 - DNA, 32 bp, Artificial Sequence
  Description of Artificial Sequenceoligo BC33
caacttcaatctagatcagaatagcagtttcc

SEQ ID NO: 46 - DNA, 33 bp, Artificial Sequence
  Description of Artificial Sequenceoligo BC34
caacttcaatctagaatcagaatagcagtttcc

SEQ ID NO: 47 - DNA, 45 bp, Artificial Sequence
  Description of Artificial Sequenceoligo BC35
ggattatacacatatgggatccgcattttccacacaagcgaaagc

(19) United States
(12) Patent Application Publication
(10) Pub. No.: US 2003/0097684 A1
(43) Pub. Date: May 22, 2003

(54) Lipid acyl hydrolases and variants thereof

(75) Inventors: Brian Carr, Raleigh, NC (US); Barbara A. Rosen, Mountain View, CA (US); Ericka R. Bermudez, Aptos, CA (US); Jon E. Ness, Redwood City, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Maxygen, Inc., Redwood City, CA

(21) Appl. No.: 10/034,937

(22) Filed: Dec. 27, 2001

Related U.S. Application Data
(60) Provisional application No. 60/260,477, filed on Jan. 8, 2001.

(51) Int. Cl. A01H 1/00; C07H 21/04; C12N 9/20; C12N 5/04; C12P 21/02

(52) U.S. Cl. 800/281; 435/198; 435/410; 435/69.1; 536/23.2

(57) ABSTRACT

The present invention provides compositions comprising lipid acyl hydrolases with improved enzymatic activity and methods for using such compositions to enhance resistance of plants to pests.

### CROSS REFERENCE OF RELATED PATENT APPLICATIONS

The present application is related to and claims priority to U.S. patent application Ser. No. (U.S. Ser. No.) 60/260,477, filed Jan. 8, 2001, which is explicitly incorporated herein by reference in its entirety and for all purposes.

### BACKGROUND OF THE INVENTION

Lipid acyl hydrolases are important enzymes that typically catalyze cleavage of long chain fatty acids. Lipid acyl hydrolases often have broad substrate specificities, and can also catalyze trans-esterification and acyl transferase reactions. Acyl hydrolases, like lipases, often catalyze reactions on substrates as broadly diverged as fatty acyl, lipid, glyco, galacto, or phopholipid substrates.

Lipid acyl hydrolases, like other lipases and esterases, act on fatty acid based substrates and are capable of catalyzing a broad array of reactions, many of which have commercial value. See, e.g., Gandhi, N., JAOCS 74:621-634 (1997). For example, hydrolases and lipases are used in the tanning of leather and flavor production in the dairy industry, as well as in the medical and waste water management industries.

A large area of use for acyl hydrolases is in the synthesis of chemical intermediates. For example, lipases and esterases can be used to perform precise steps in the biochemical synthesis of chiral intermediates. Hirohara, H., et al., Biosci. Biotechnol. Biochem., 62(1):1-9 (1998) describes use of hydrolases to perform stereoselective steps at several stages of the synthesis of insecticides. Marchalin, S., et al., Heterocycles 48(9):1943-1958 (1998) describes the use of hydrolases in biotransformation. In addition, Stephen C. Taylor reviews potential uses for lipases (and therefore acyl hydrolases) in the biocatalysis industry. See, Taylor, Industrial Bioconversions for Chiral Molecules, Issues and Developments in MEDICINAL CHEMISTRY: TODAY AND TOMORROW (Yamazaki, M., ed.) (1996). Thus, acyl hydrolases are valuable in many of these industries.

Acyl hydrolases, like lipases, are capable of catalyzing reactions in addition to the hydrolysis of fatty acyl, lipid, glyco, galacto, or phopholipid substrates. Under the correct conditions, these enzymes can be made to work in reverse, catalyzing trans-esterification reactions to create ester linkages. For example, Yahya, A.R.M., et al., Enzyme and Microbial Technology 23: 438-450 (1998) describes some of the many ester synthesis reactions catalyzed by lipases. Acyl hydrolases have commercial value in facilitating such reactions, as well as novel reactions not described therein. By substitution of alcohols for water as the acceptor molecule, these enzymes can also perform acyl transferase functions, i.e., by transferring the cleaved fatty acid to methanol or other alcohols.

Acyl hydrolases have been identified in many plant species. See, e.g., Huang, A., et al., Lipases in THE BIOCHEMISTRY OF PLANTS, (1987). The most well characterized plant lipid acyl hydrolase is Patatin. Patatin is actually a mixture of isozymes (Racusen, D., Can. J. Bot. 62:1640-1644 (1984)) produced in potato tubers and leaves by a multi-gene family (Mignery et al., Gene 62:27-44 (1988)). Patatin cDNAs have been cloned and sequenced. See, e.g., Mignery et al., Nuc. Acids. Res. 12: 7987-8000 (1984)). The substrate specificity of Patatin for a broad range of substrates has been tested. See, Galliard T., et al., Biochem J. 121:379-390 (1971)). Patatin has many properties, including those typical of an acyl hydrolase (such as the ability to perform acyl transferase reactions). See, Galliard and Dennis, Phytochemistry 13: 2463-2468 (1974).

Pentin is a lipid acyl hydrolase isolated from the seeds of Pentaclethra macroloba. See, U.S. Pat. No. 6,057,491. A homologue of Pentin has been cloned and sequenced from maize. See, U.S. Pat. Nos. 5,824,864 and 5,882,668. Lipid acyl hydrolases have been isolated from other plants, including soybean, Arabidopsis, and sorgum. Indeed, the sequences of many cDNAs with homology to Pentin and/or Patatin can be identified by computer search of public DNA sequence databases such as Genbank.

Pentin has been shown to have insecticidal activity. See, U.S. Pat. No. 6,057,491. Patatin has also been shown to have insecticidal activity. See, U.S. Pat. No. 5,743,477. In the case of Patatin, it has been demonstrated that lipid acyl hydrolase activity is required for insecticidal activity. See, U.S. Pat. No. 5,743,477.

Improvement of lipid acyl hydrolase activity is useful to improve the insecticidal activity of the enzymes. These and other advantages are provided by the present application.

### SUMMARY OF THE INVENTION

The present invention provides for isolated nucleic acids encoding lipid acyl hydrolases. For instance, the invention provides isolated nucleic acids comprising a polynucleotide encoding a polypeptide, wherein the polypeptide is at least 70% identical to a polypeptide selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38 and SEQ ID NO:40, with the proviso that the polypeptide is not SEQ ID NO:42 or SEQ ID NO:43. In some embodiments, the polypeptides exhibit lipid acyl hydrolase and/or insecticidal activity. In some aspects, the lipid acyl hydrolase polypeptide has an activity at least 20%, or at least 200%, or at least 1,000% of the lipid acyl hydrolase activity of SEQ ID NO:41. In some aspects of the invention, the isolated nucleic acid, for example, can be selected from the following: SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37 and SEQ ID NO:39. In some aspects of the invention, the polypeptides are selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, and SEQ ID NO:40. In some aspects, the nucleic acids of the invention are in a vector.

The present invention also provides for isolated nucleic acids comprising a polynucleotide encoding a polypeptide with lipid acyl hydrolase and/or insecticidal activity, wherein the nucleic acid specifically hybridizes under stringent conditions to a probe polynucleotide selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37 and SEQ ID NO:39, with the proviso that the lipid acyl hydrolase polynucleotide does not encode SEQ ID NO:42 or SEQ ID NO:43.

The present invention also provides an isolated nucleic acid comprising a polynucleotide encoding a polypeptide with lipid acyl hydrolase and/or insecticidal activity, wherein the polypeptide is at least 70% identical to SEQ ID NO:41 and the polypeptide comprises at least one of the following alterations: D240G, I241T, I241N or S49P.

The present invention also provides isolated nucleic acids of at least 20 nucleotides in length encoding an amino acid sequence or subsequence of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38 and SEQ ID NO:40, with the proviso that the nucleic acid is not an amino acid subsequence of SEQ ID NO:42 or SEQ ID NO:43. In some aspects, these nucleic acids encode amino acid sequences with lipid acyl hydrolase and/or insecticidal activity. In some aspects, the lipid acyl hydrolase activity is improved over the activity of SEQ ID NO:43.

In some aspects, the isolated nucleic acids of the invention further comprise a promoter operably linked to the polynucleotide encoding the lipid acyl hydrolase. For example, the promoter can be tissue-preferred and can be inducible or constitutive.

The present invention also provides an isolated polypeptide at least 70% identical to a polypeptide selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38 and SEQ ID NO:40, with the proviso that the polypeptide is not SEQ ID NO:42 or SEQ ID NO:43. In some embodiments, the polypeptides have lipid acyl hydrolase and/or insecticidal activity. The present invention also provides isolated polypeptides with lipid acyl hydrolase activity, wherein the polypeptides are at least 70% identical to SEQ ID NO:41 and the polypeptides comprise at least one of the following alterations: D240G, I241T, I241N or S49P. The invention also provides for antibodies capable of binding the lipid acyl hydrolases of the invention.

The invention also provides a plant comprising a nucleic acid of the invention, including, e.g., a recombinant expression cassette comprising a promoter operably linked to a polynucleotide encoding a polypeptide with lipid acyl hydrolase and/or insecticidal activity, wherein the polypeptide is at least 70% identical to a polypeptide elected from the group comprising SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38 and SEQ ID NO:40, with the proviso that the polypeptide is not SEQ ID NO:42 or SEQ ID NO:43. The invention also provides plants comprising a polynucleotide encoding a polypeptide with lipid acyl hydrolase and/or insecticidal activity, wherein the polypeptide is at least 70% identical to SEQ ID NO:41 and the polypeptide comprises at least one of the following alterations: D240G, I241T, I241N or S49P. Plants of the invention include, e.g., maize, soybean potato and cotton. In some aspects, the lipid acyl hydrolase polypeptide has an activity at least 20%, or at least 200%, or at least 1,000% of the lipid acyl hydrolase activity of SEQ ID NO:41.

The present invention also provides a method of enhancing plant resistance to a pest, the method comprising: (a) introducing into the plant a recombinant expression cassette comprising a promoter operably linked to a polynucleotide encoding a polypeptide at least 70% identical to a polypeptide selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38 and SEQ ID NO:40, with the proviso that the polypeptide is not SEQ ID NO:42 or SEQ ID NO:43, and (b) selecting a plant with enhanced resistance. The invention also provides for methods of enhancing plant resistance to a pest by introducing into the plant, a recombinant expression cassette comprising a promoter operably linked to a polynucleotide encoding a polypeptide with lipid acyl hydrolase activity, wherein the polypeptide is at least 70% identical to SEQ ID NO:41 and the polypeptide comprises at least one of the following alterations: D240G, I241T, I241N or S49P. In some aspects, the methods are used to enhance the resistance of maize, soybean, potato or cotton.

### Definitions

The phrase “nucleic acid sequence” refers to a single or double-stranded polymer of deoxyribonucleotide or ribonucleotide bases read from the 5′ to the 3′ end. It includes chromosomal DNA, self-replicating plasmids, infectious polymers of DNA or RNA and DNA or RNA that performs a primarily structural role.

The term “encoding” refers to a polynucleotide sequence encoding one or more amino acids. The term does not require a start or stop codon. An amino acid sequence can be encoded in any one of six different reading frames provided by a polynucleotide sequence.

The term “promoter” refers to regions or sequence located upstream and/or downstream from the start of transcription and which are involved in recognition and binding of RNA polymerase and other proteins to initiate transcription.

A “vector” refers to a polynucleotide, which when independent of the host chromosome, is capable replication in a host organism. Preferred vectors include plasmids and typically have an origin of replication. Vectors can comprise, e.g., transcription and translation terminators, transcription and translation initiation sequences, and promoters useful for regulation of the expression of the particular nucleic acid.

The term “plant” includes whole plants, shoot vegetative organs/structures (e.g. leaves, stems and tubers), roots, flowers and floral organs/structures (e.g. bracts, sepals, petals, stamens, carpels, anthers and ovules), seed (including embryo, endosperm, and seed coat) and fruit (the mature ovary), plant tissue (e.g. vascular tissue, ground tissue, and the like) and cells (e.g. guard cells, egg cells, trichomes and the like), and progeny of same. The class of plants that can be used in the method of the invention is generally as broad as the class of higher and lower plants amenable to transformation techniques, including angiosperms (monocotyledonous and dicotyledonous plants), gymnosperms, ferns, and multicellular algae. It includes plants of a variety of ploidy levels, including aneuploid, polyploid, diploid, haploid and hemizygous.

A polynucleotide sequence is “heterologous to” an organism or a second polynucleotide sequence if it originates from a foreign species, or, if from the same species, is modified from its original form. For example, a promoter operably linked to a heterologous coding sequence refers to a coding sequence from a species different from that from which the promoter was derived, or, if from the same species, a coding sequence which is not naturally associated with the promoter (e.g. a genetically engineered coding sequence or an allele from a different ecotype or variety).

“Recombinant” refers to a human manipulated polynucleotide or a copy or complement of a human manipulated polynucleotide. For instance, a recombinant expression cassette comprising a promoter operably linked to a second polynucleotide may include a promoter that is heterologous to the second polynucleotide as the result of human manipulation (e.g., by methods described in Sambrook et al., Molecular Cloning - A Laboratory Manual, Cold Spring Harbor Laboratory, Cold Spring Harbor, New York, (1989) or Current Protocols in Molecular Biology Volumes 1-3, John Wiley & Sons, Inc. (1994-1998)) of an isolated nucleic acid comprising the expression cassette. In another example, a recombinant expression cassette may comprise polynucleotides combined in such a way that the polynucleotides are extremely unlikely to be found in nature. For instance, human manipulated restriction sites or plasmid vector sequences may flank or separate the promoter from the second polynucleotide. One of skill will recognize that polynucleotides can be manipulated in many ways and are not limited to the examples above.

A polynucleotide “exogenous to” an individual plant is a polynucleotide which is introduced into the plant by any means other than by a sexual cross. Examples of means by which this can be accomplished are described below, and include Agrobacterium-mediated transformation, biolistic methods, electroporation, and the like. Such a plant containing the exogenous nucleic acid is referred to here as a T₁ (e.g. in Arabidopsis by vacuum infiltration) or R₀ (for plants regenerated from transformed cells in vitro) generation transgenic plant. Transgenic plants that arise from sexual cross or by selfing are descendants of such a plant.

A “lipid acyl hydrolase nucleic acid” or “lipid acyl hydrolase polynucleotide sequence” of the invention is a polynucleotide sequence or subsequence (e.g., odd numbered sequences from SEQ ID NO:1 to SEQ ID NO:39) which, encodes a lipid acyl hydrolase polypeptide (e.g., even numbered sequences from SEQ ID NO:2 to SEQ ID NO:40) with lipid acyl hydrolase activity. “Lipid acyl hydrolase” refers to a polypeptide capable of enzymatically cleaving long chain fatty acids and typically also has the ability to perform acyl transferase (e.g., the reverse) reactions. See, Galliard and Dennis, Phytochemistry 13: 2463-2468 (1974). Thus lipid acyl hydrolase polypeptides inherently have “lipid acyl hydrolase activity.” Typical substrates of lipid acyl hydrolases include glyco- and phospho- lipids such as monogalactosyldiacylglycerol, acylsterylglucoside, phosphatidylcholine and lysophosphophatidylcholine.

“Lipid acyl hydrolase nucleic acids” or “lipid acyl hydrolase polynucleotide sequences” also include polynucleotides of at least about 10, preferably about 15, more preferably about 20, more preferably about 30 and most preferably about 50 nucleotides in length that encode subsequences of the above-described lipid acyl polypeptides (e.g., even numbered sequences from SEQ ID NO:2 to SEQ ID NO:40) that are not comprised in SEQ ID NO:43. Lipid acyl hydrolase polynucleotides are typically less than about 10,000 nucleotides, preferably less than 5,000 nucleotides and sometimes less than 1,000 or 500 or 100 nucleotides in length. Exemplary subsequences comprise the following alterations of SEQ ID NO:41: D240G, I241T, I241N and/or S49P.

Some lipid hydrolases of the invention exhibit improved lipid acyl hydrolase activity as compared to the lipid acyl hydrolase displayed in SEQ ID NO:41, SEQ ID NO:42 or SEQ ID NO:43, in the assays described herein. A typical lipid acyl hydrolase enzymatic assay consists of measuring the hydrolysis of p-nitrophenyl caprylate spectrophotometrically, as described herein. Typically, lipid acyl hydrolases of the invention exhibit an improvement of lipid acyl hydrolase activity at least about 150% of the lipid acyl hydrolase of SEQ ID NO:41, more typically at least 200% of the activity of SEQ ID NO:41, usually at least about 500% of the activity of SEQ ID NO:41, preferably at least 1,000% of activity of SEQ ID NO:41, and more preferably 10,000% of activity of SEQ ID NO:41.

Other lipid acyl hydrolase polypeptides of the invention have lower lipid acyl hydrolase activity than SEQ ID NO:43. Typically, these lipid acyl hydrolases of the invention exhibit substantially the same activity as SEQ ID NO:43. The polypeptides of the invention, however, can exhibit less than 70%, sometimes less than 50% and even less than 20% or less than 10% of the activity of SEQ ID NO:43.

The polynucleotides of the invention encode polypeptides that are preferably at least 70% identical, more preferably at least 80% identical and most preferably at least 90% identical to SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40 or SEQ ID NO:42, with the proviso that the lipid acyl hydrolase polypeptide is not the wild type pentin protein (SEQ ID NO:41). In some cases, the encoded polypeptides have lipid acyl hydrolase and/or insecticidal activity.

A polypeptide exhibits “insecticidal activity” if the polypeptide has an LC50 of less than or equal to about 1 g/ml, more preferably 100 mg/ml, more preferably 10 mg/ml, and even more preferably 1 mg/ml of insect food versus a particular insect according to the insecticidal assays provided herein.

In the case of both expression of transgenes and inhibition of endogenous genes (e.g., by antisense, or co-suppression) one of skill will recognize that the inserted polynucleotide sequence need not be identical, but may be only “substantially identical” to a sequence of the gene from which it was derived. As explained below, these substantially identical variants are specifically covered by the term “lipid acyl hydrolase nucleic acid.”

In the case where the inserted polynucleotide sequence is transcribed and translated to produce a functional polypeptide, one of skill will recognize that because of codon degeneracy a number of polynucleotide sequences will encode the same polypeptide. These variants are specifically covered by the terms “lipid acyl hydrolase nucleic acid,” “lipid acyl hydrolase polynucleotide” and their equivalents. In addition, the terms specifically include those full length sequences substantially identical (determined as described below) with an lipid acyl hydrolase polynucleotide sequence and that encode proteins that retain the function of the lipid acyl hydrolase polypeptide (e.g., resulting from conservative substitutions of amino acids in the lipid acyl hydrolase polypeptide).

As used herein, an “antibody” refers to a protein consisting of one or more polypeptide substantially or partially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. A typical immunoglobulin (antibody) structural unit is known to comprise a tetramer. Each tetramer is composed of two identical pairs of polypeptide chains, each pair having one “light” (about 25 kD) and one “heavy” chain (about 50-70 k). The N-terminus of each chain defines a variable region of about 100 to 1 10 or more amino acids primarily responsible for antigen recognition. The terms variable light chain (VL) and variable heavy chain (VH) refer to these light and heavy chains respectively. Antibodies exist as intact immunoglobulins or as a number of well characterized fragments produced by digestion with various peptidases. Thus, for example, pepsin digests an antibody below the disulfide linkages in the hinge region to produce F(ab)′2, a dimer of Fab which itself is a light chain joined to VH-CH1 by a disulfide bond. The F(ab)′2 may be reduced under mild conditions to break the disulfide linkage in the hinge region thereby converting the (Fab′)2 dimer into an Fab′ monomer. The Fab′ monomer is essentially an Fab with part of the hinge region (see, Fundamental Immunology, W. E. Paul, ed., Raven Press, N.Y. (1993), for a more detailed description of other antibody fragments). While various antibody fragments are defined in terms of the digestion of an intact antibody, one of skill will appreciate that such Fab′ fragments may be synthesized de novo either chemically or by utilizing recombinant DNA methodology. Thus, the term antibody, as used herein also includes antibody fragments either produced by the modification of whole antibodies or synthesized de novo using recombinant DNA methodologies. Antibodies include single chain antibodies, including single chain Fv (sFv) antibodies in which a variable heavy and a variable light chain are joined together (directly or through a peptide linker) to form a continuous polypeptide.

“Pests” include, but are not limited to all types of insects and nematodes, as well as, viruses, bacteria, nematodes, fungi or the like. For example, economically important phytophagous insects include corn rootworms e.g., Diabrotica spp., especially D. barberi (northern corn rootworm), D. undecimpunctata (cucumber beetles) and D. virgifera (western corn rootworm)); potato beetles (Leptinotarsa spp., especially L. decemlineata), armyworms (Spodoptera spp., especially Spodoptera frugiperda), borers (Ostrinia spp. and Diatraea spp., especially Ostrinia nubilalis), cutworms (especially Agrotisipsilon), wireworms (Elateridae, Agriotes spp.), earworms (Heliothis spp., especially Heliothis zea) and aphids (Rhopalosiphum maydis and Schizaphis graminum). Further examples of insect pests are described in, e.g., PCT WO 98/54327.

Two nucleic acid sequences or polypeptides are said to be “identical” if the sequence of nucleotides or amino acid residues, respectively, in the two sequences is the same when aligned for maximum correspondence as described below. The terms “identical” or percent “identity,” in the context of two or more nucleic acids or polypeptide sequences, refer to two or more sequences or subsequences that are the same or have a specified percentage of amino acid residues or nucleotides that are the same, when compared and aligned for maximum correspondence over a comparison window, as measured using one of the following sequence comparison algorithms or by manual alignment and visual inspection. When percentage of sequence identity is used in reference to proteins or peptides, it is recognized that residue positions that are not identical often differ by conservative amino acid substitutions, where amino acids residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated according to, e.g., the algorithm of Meyers & Miller, Computer Applic. Biol. Sci. 4:11-17 (1988) e.g., as implemented in the program PC/GENE (Intelligenetics, Mountain View, Calif., USA).

The term “substantial identity” of polynucleotide sequences means that a polynucleotide comprises a sequence that has at least 25% sequence identity. Alternatively, percent identity can be any integer from 25% to 100%. More preferred embodiments include at least: 25%, 30%, 35%, 40%, 45%, 50%, 55%, 60%, 65%, 70%, 75%, 80%, 85%, 90%, 95%, or 99%. compared to a reference sequence using the programs described herein; preferably BLAST using standard parameters, as described below. One of skill will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of at least 40%. Preferred percent identity of polypeptides can be any integer from 40% to 100% (e.g., 40%,41%, 42%, 43%, etc.). More preferred embodiments include at least 60%, 65%, 70%, 75%, 80%, 85%, 90%, 95%, or 99%. Polypeptides which are “substantially similar” share sequences as noted above except that residue positions which are not identical may differ by conservative amino acid changes. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, aspartic acid-glutamic acid, and asparagine-glutamine.

One of skill in the art will recognize that two polypeptides can also be “substantially identical” if the two polypeptides are immunologically similar. Thus, overall protein structure may be similar while the primary structure of the two polypeptides display significant variation. Therefore a method to measure whether two polypeptides are substantially identical involves measuring the binding of monoclonal or polyclonal antibodies to each polypeptide. Two polypeptides are substantially identical if the antibodies specific for a first polypeptide bind to a second polypeptide with an affinity of at least one third of the affinity for the first polypeptide.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection.

One example of a useful algorithm is PILEUP. PILEUP creates a multiple sequence alignment from a group of related sequences using progressive, pairwise alignments to show relationship and percent sequence identity. It also plots a tree or dendogram showing the clustering relationships used to create the alignment. PILEUP uses a simplification of the progressive alignment method of Feng & Doolittle, J. Mol. Evol. 35:351-360 (1987). The method used is similar to the method described by Higgins & Sharp, CABIOS 5:151-153 (1989). The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. For example, a reference sequence can be compared to other test sequences to determine the percent sequence identity relationship using the following parameters: default gap weight (3.00), default gap length weight (0.10), and weighted end gaps.

Another example of algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are extended in both directions along each sequence for as far as the cumulative alignment score can be increased. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLAST program uses as defaults a word length (W) of 11, the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine) can be modified to yield a functionally identical molecule. Accordingly, each silent variation of a nucleic acid which encodes a polypeptide is implicit in each described sequence.

As to amino acid sequences, one of skill will recognize that individual substitutions, in a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art.

The following six groups each contain amino acids that are conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V); and

6) Phenylalanine (F), Tyrosine (Y), Tryptophan (W). (see, e.g., Creighton, Proteins (1984)).

An indication that two nucleic acid sequences or polypeptides are substantially identical is that the polypeptide encoded by the first nucleic acid is immunologically cross reactive with the antibodies raised against the polypeptide encoded by the second nucleic acid. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below.

The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA).

The phrase “stringent hybridization conditions” refers to conditions under which a probe will hybridize to its target subsequence, typically in a complex mixture of nucleic acid, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology-Hybridization with Nucleic Probes, “Overview of principles of hybridization and the strategy of nucleic acid assays” (1993). Generally, highly stringent conditions are selected to be about 5-10° C. lower than the thermal melting point (Tₘ) for the specific sequence at a defined ionic strength pH. Low stringency conditions are generally selected to be about 15-30° C. below the Tₘ. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Hybridization conditions are typically those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is at least two times background, preferably 10 times background hybridization. For the purposes of this disclosure, stringent conditions for such RNA-DNA hybridizations are those which include at least one wash in 0.2X SSC at 63° C. for 20 minutes, or equivalent conditions. Genomic DNA or cDNA comprising genes of the invention can be identified using the polynucleotides exlicitly disclosed herein (e..g, odd numbered SEQ ID NOs from 1-39), or fragments thereof of at least about 100 nucleotides, under stringent conditions, which for purposes of this disclosure, include at least one wash (usually 2) in 0.2X SSC at a temperature of at least about 50° C., usually about 55° C., and sometimes 60 ° C., for 20 minutes, or equivalent conditions.

Nucleic acids that do not hybridize to each other under stringent conditions are still substantially identical if the polypeptides that they encode are substantially identical. This occurs, for example, when a copy of a nucleic acid is created using the maximum codon degeneracy permitted by the genetic code. In such cases, the nucleic acids typically hybridize under moderately stringent hybridization conditions.

In the present invention, genomic DNA or cDNA comprising lipid acyl hydrolase nucleic acids of the invention can be identified in standard Southern blots under stringent conditions using the nucleic acid sequences disclosed here. For the purposes of this disclosure, suitable stringent conditions for such hybridizations are those which include a hybridization in a buffer of 40% formamide, 1 M NaCl, 1% SDS at 37° C., and at least one wash in 0.2X SSC at a temperature of at least about 50° C., usually about 55° C. to about 60° C., for 20 minutes, or equivalent conditions. A positive hybridization is at least twice background. Those of ordinary skill will readily recognize that alternative hybridization and wash conditions can be utilized to provide conditions of similar stringency.

Nucleic acids that do not hybridize to each other under stringent conditions are still substantially identical if the polypeptides that they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1X SSC at 45° C. A positive hybridization is at least twice background. Those of ordinary skill will readily recognize that alternative hybridization and wash conditions can be utilized to provide conditions of similar stringency.

A further indication that two polynucleotides are substantially identical is if the reference sequence, amplified by a pair of oligonucleotide primers, can then be used as a probe under stringent hybridization conditions to isolate the test sequence from a cDNA or genomic library, or to identify the test sequence in, e.g., an RNA gel or DNA gel blot hybridization analysis.

### BRIEF DESCRIPTION OF THE DRAWINGS

FIG. 1A and B is a graphical representation of acyl hydrolase assays results from multiple (typically sixteen; see Table 1) independent growths of each clone. For comparison, wild type clones (typically 16 total) were grown in same plate. Error bars represent standard deviation. The white (WT) bar closest to the left of the grey bars represents the average of activity for wild type clones grown in same plate. Dashed line represents 3 standard deviations above mean wild type activity. PIP1 mean activity was 928 with SD=153 and PIP-55 mean activity was 475 with SD=58; the top of this bar is removed to save space.

FIG. 2 displays an amino acid sequence alignment of lipid acyl hydrolase polypeptides with improved enzymatic activity.

### DESCRIPTION OF THE SPECIFIC EMBODIMENTS

The present invention provides lipid acyl hydrolase polypeptides with improved enzymatic activity. The invention provides individual sequences that have enhanced enzymatic activity over the wild type (SEQ ID NO:41) pentin protein.

The invention also provides an analysis of specific amino acid residues within the pentin protein that can be modified to modulate lipid acyl hydrolase activity. The specific sequences are provided below.

The present invention also provides methods of improving resistance of plants to pests. In particular, resistance of plants to insects can be enhanced by introducing improved lipid acyl hydrolase polypeptides into plants.

Polypeptides of the invention

Polypeptides of the invention exhibit the same or altered lipid acyl hydrolase and/or insecticidal activity compared to that of the wild type pentin protein, in accordance with the activity assays described herein. In some cases, the polypeptides of the invention exhibit improved activity compared to the wild type pentin protein. Without intending to limit the invention to a particular mechanism of operation, it is believed that changing certain residues of the pentin protein results in improved catalytic activity of the protein. Kinetic studies of the improved enzymatic activity of the polypeptides of the invention demonstrate that the enzymes' Vₘₐₓ is increased and the Kₘ is substantially the same as wild type. Therefore, it is believed that k_{cat}, i.e., release of the cleaved fatty acid, is the step affected in the polypeptides with improved activity.

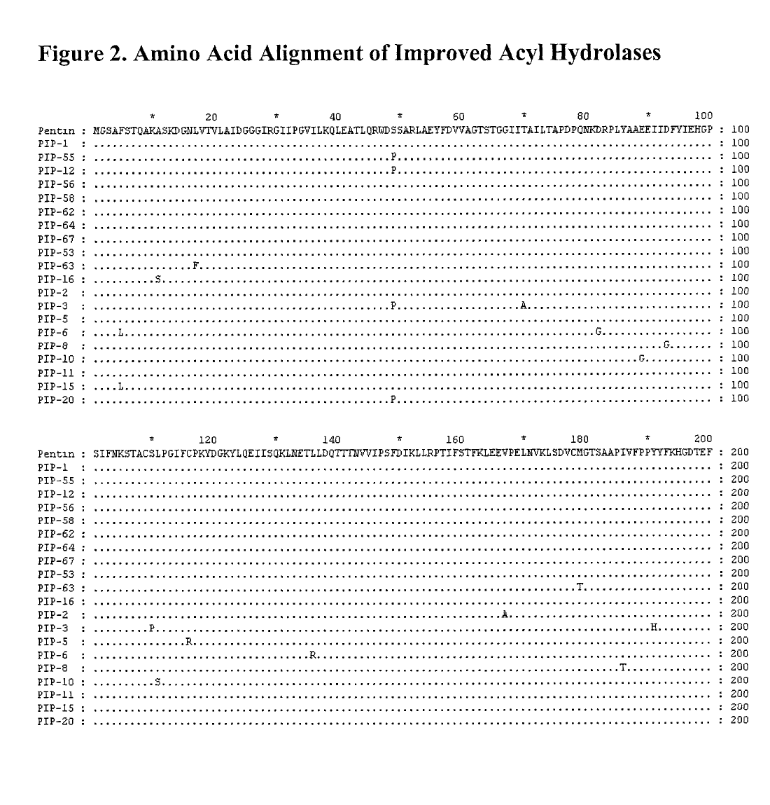

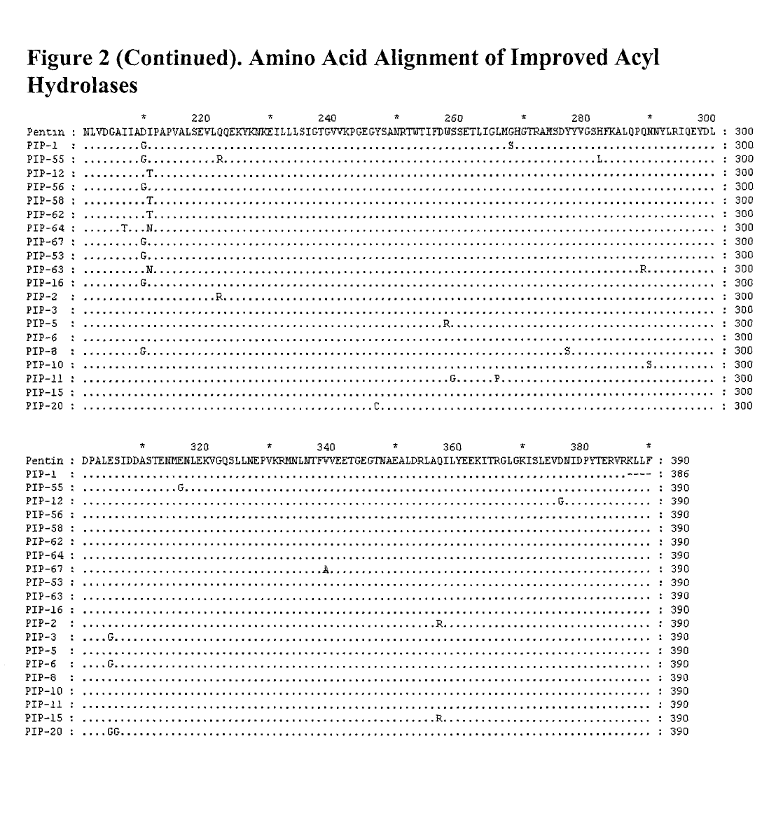

An analysis of the sequences of the polypeptides of the invention provides certain themes that indicate important residues for the improvement of activity of lipid acyl hydrolases. FIG. 2, below, depicts differences of exemplary polypeptides from the wild type pentin protein.

The polypeptides displayed in FIG. 2 are ordered by decreasing activity. Each of the peptides of the invention in FIG. 2 exhibited at least about 50% greater lipid acyl hydrolase activity than that of the truncated wildtype pentin (SEQ ID NO:41) in the assays described herein. Without intending to limit the invention to a particular mechanism, the aspartic acid residue (D) at position 240 of the pentin protein (SEQ ID NO:43) and isoleucine (I) at position 241, appear to play a role in enhancing the activity of pentin and improving lipid acyl hydrolase activity. For example, the twelve most active polypeptides in FIG. 2 have an alteration at one of the two above-referenced positions. As will be noted in the figure, alterations at these positions were either D240G (i.e., the aspartic acid at position 240 of SEQ ID NO:1 replaced with a glycine (G)), I241T or I241N in the twelve most active polypeptides. Of further note, S49P appears at a notable frequency in the polypeptides of the invention. Other alterations are also apparent from FIG. 2. Therefore, lipid acyl hydrolases with these changes, or combinations thereof, will have increased activity over the wild type pentin protein

The combination of alterations in the polypeptides of the invention result in a variety of levels of enzymatic activity. Thus, combinations of different alterations, which individually provide positive or negative effects, result in the ultimate variation in activity found in the polypeptides of the invention. For example, combinations of positive and negative (i.e., inhibitory) alterations can lead to improved activity over wild type pentin activity. In some cases, it is possible that alterations that produce a negative effect when added singly can have a positive, synergistic effect when combined with other alterations.

Purification of lipid acyl hydrolase polypeptides

Either naturally occurring or recombinant lipid acyl hydrolase polypeptides can be purified for use in functional assays. Naturally occurring lipid acyl hydrolase polypeptides can be purified, e.g., from plant tissue and any other source of a lipid acyl hydrolase. Recombinant lipid acyl hydrolase polypeptides can be purified from any suitable expression system.

The lipid acyl hydrolase polypeptides may be purified to substantial purity by standard techniques, including selective precipitation with such substances as ammonium sulfate; column chromatography, immunopurification methods, and others (see, e.g., Scopes, Protein Purification: Principles and Practice (1982); U.S. Pat. No. 4,673,641; Sambrook et al., Molecular Cloning - A Laboratory Manual, Cold Spring Harbor Laboratory, Cold Spring Harbor, New York, (1989) or Current Protocols in Molecular Biology Volumes 1-3, John Wiley & Sons, Inc. (1994-1998)(Ausubel et al.)).

A number of procedures can be employed when recombinant lipid acyl hydrolase polypeptides are being purified. For example, proteins having established molecular adhesion properties can be reversibly fused to the lipid acyl hydrolase polypeptides. With the appropriate ligand, the lipid acyl hydrolase polypeptides can be selectively adsorbed to a purification column and then freed from the column in a relatively pure form. The fused protein can then be removed by enzymatic activity. Finally the lipid acyl hydrolase polypeptides could be purified using immunoaffinity columns.

Cross-reactivity determinations

Immunoassays in a competitive binding format can be used to identify polypeptide sequences with cross reactivity to an antibody raised to a particular polypeptide or epitope of the invention. For example, a protein at least partially encoded by an odd numbered sequence between SEQ ID NO:1 and SEQ ID NO:39, or an immunogenic region thereof, can be immobilized to a solid support. Other proteins such as the native pentin polypeptide or modifications or fragments thereof, are added to the assay so as to compete for binding of the antisera to the immobilized antigen. The ability of the added proteins to compete for binding of the antisera to the immobilized protein is compared to the ability of the particular polypeptide of the invention (e.g., even-numbered sequences friom SEQ ID NO:2 to SEQ ID NO:40) to compete with itself. The percent cross-reactivity for the above proteins is calculated, using standard calculations. Those antisera with less than 10% crossreactivity with each of the added proteins listed above are selected and pooled. The cross-reacting antibodies are optionally removed from the pooled antisera by immunoabsorption with the added considered proteins, e.g., distantly related homologs, or other polypeptide sequences homologous to the polypeptides of the invention (e.g., wild type pentin).

The immunoabsorbed and pooled antisera are then used in a competitive binding immunoassay as described below to compare a second protein, thought to be perhaps an allele or polymorphic variant of the particular lipid acyl hydrolase, to the immunogen protein. In order to make this comparison, the two proteins are each assayed at a wide range of concentrations and the amount of each protein required to inhibit 50% of the binding of the antisera to the immobilized protein is determined. If the amount of the second protein required to inhibit 50% of binding is less than 10 times the amount of the lipid acyl hydrolase polypeptide of the invention that is required to inhibit 50% of binding, then the second protein is said to specifically bind to the polyclonal antibodies generated to the respective lipid acyl hydrolase immunogen.

Competitive immunoassay formats

In competitive assays, the amount of the lipid acyl hydrolase present in the sample is measured indirectly by measuring the amount of known, added (exogenous) lipid acyl hydrolase displaced (competed away) from an anti-lipid acyl hydrolase antibody by the unknown lipid acyl hydrolase present in a sample. In one competitive assay, a known amount of the lipid acyl hydrolase is added to a sample and the sample is then contacted with an antibody that specifically binds to the lipid acyl hydrolase. The amount of exogenous lipid acyl hydrolase bound to the antibody is inversely proportional to the concentration of the lipid acyl hydrolase present in the sample. In a particularly preferred embodiment, the antibody is immobilized on a solid substrate. The amount of lipid acyl hydrolase bound to the antibody may be determined either by measuring the amount of lipid acyl hydrolase present in a lipid acyl hydrolase /antibody complex, or alternatively by measuring the amount of remaining uncomplexed protein. The amount of lipid acyl hydrolase may be detected by providing a labeled lipid acyl hydrolase molecule.

A hapten inhibition assay is another preferred competitive assay. In this assay the known lipid acyl hydrolase is immobilized on a solid substrate. A known amount of anti-lipid acyl hydrolase antibody is added to the sample, and the sample is then contacted with the immobilized lipid acyl hydrolase. The amount of anti-lipid acyl hydrolase antibody bound to the known immobilized lipid acyl hydrolase is inversely proportional to the amount of lipid acyl hydrolase present in the sample. Again, the amount of immobilized antibody may be detected by detecting either the immobilized fraction of antibody or the fraction of the antibody that remains in solution. Detection may be direct where the antibody is labeled or indirect by the subsequent addition of a labeled moiety that specifically binds to the antibody as described above.

Lipid acyl hydrolase nucleic acids

Nucleic acids of the invention generally comprise all or part of a polynucleotide encoding a lipid acyl hydrolase polypeptide of the invention. In some preferred embodiments, the nucleic acids encode at least one of the alterations from the native pentin protein sequence, as noted in FIG. 2.

Nucleic acids of the invention also encompass nucleic acid probes. Probes are useful, for instance, to detect differences between native pentin polynucleotides and polynucleotides encoding alterations in the pentin sequence that give rise to improved or altered enzymatic and/or insecticidal activity. Probes can be of any length useful to detect a desired polynucleotide. For example, those of skill in the art will recognize that probes can be designed to selectively hybridize to a polynucleotide encoding the D240G alteration but not hybridize to the native pentin polynucleotide sequence encoding the aspartic acid residue at position 240.

Lipid acyl hydrolase polynucleotides of the invention can be readily modified using methods that are well known in the art to improve or alter lipid acyl hydrolase and/or insecticidal activity. A variety of diversity generating protocols are available and described in the art. The procedures can be used separately, and/or in combination to produce one or more variants of a nucleic acid or set of nucleic acids, as well variants of encoded proteins. Individually and collectively, these procedures provide robust, widely applicable ways of generating diversified nucleic acids and sets of nucleic acids (including, e.g., nucleic acid libraries) useful, e.g., for the engineering or rapid evolution of nucleic acids, proteins, pathways, cells and/or organisms with new and/or improved characteristics.

While distinctions and classifications are made in the course of the ensuing discussion for clarity, it will be appreciated that the techniques are often not mutually exclusive. Indeed, the various methods can be used singly or in combination, in parallel or in series, to access diverse sequence variants.

The result of any of the diversity generating procedures described herein can be the generation of one or more nucleic acids, which can be selected or screened for nucleic acids that encode proteins with or which confer desirable properties. Following diversification by one or more of the methods herein, or otherwise available to one of skill, any nucleic acids that are produced can be selected for a desired activity or property, e.g. lipid acyl hydrolase activity. This can include identifying any activity that can be detected, for example, in an automated or automatable format, by any of the assays in the art, e.g., by assaying the hydrolysis of p-nitrophenyl caprylate, as described herein. A variety of related (or even unrelated) properties can be evaluated, in serial or in parallel, at the discretion of the practitioner.

Descriptions of a variety of diversity generating procedures for generating modified lipid acyl hydrolase nucleic acid sequences of the invention are found the following publications and the references cited therein: Stemmer, et al. (1999) “Molecular breeding of viruses for targeting and other clinical properties” Tumor Targeting 4:1-4; Ness et al. (1999) “DNA Shuffling of subgenomic sequences of subtilisin” Nature Biotechnology 17:893-896; Chang et al. (1999) “Evolution of a cytokine using DNA family shuffling” Nature Biotechnology 17:793-797; Minshull and Stemmer (1999) “Protein evolution by molecular breeding” Current Opinion in Chemical Biology 3:284-290; Christians et al. (1999) “Directed evolution of thymidine kinase for AZT phosphorylation using DNA family shuffling” Nature Biotechnology 17:259-264; Crameri et al. (1998) “DNA shuffling of a family of genes from diverse species accelerates directed evolution” Nature 391:288-291; Crameri et al. (1997) “Molecular evolution of an arsenate detoxification pathway by DNA shuffling,” Nature Biotechnology 15:436-438; Zhang et al. (1997) “Directed evolution of an effective fucosidase from a galactosidase by DNA shuffling and screening” Proc. Natl. Acad. Sci. USA 94:4504-4509; Patten et al. (1997) “Applications of DNA Shuffling to Pharmaceuticals and Vaccines” Current Opinion in Biotechnology 8:724-733; Crameri et al. (1996) “Construction and evolution of antibody-phage libraries by DNA shuffling” Nature Medicine 2:100-103; Crameri et al. (1996) “Improved green fluorescent protein by molecular evolution using DNA shuffling” Nature Biotechnology 14:315-319; Gates et al. (1996) “Affinity selective isolation of ligands from peptide libraries through display on a lac repressor ‘headpiece dimer’” Journal of Molecular Biology 255:373-386; Stemmer (1996) “Sexual PCR and Assembly PCR” The Encyclopedia of Molecular Biology. VCH Publishers, New York. pp.447-457; Crameri and Stemmer (1995) “Combinatorial multiple cassette mutagenesis creates all the permutations of mutant and wildtype cassettes” BioTechniques 18:194-195; Stemmer et al., (1995) “Single-step assembly of a gene and entire plasmid form large numbers of oligodeoxy-ribonucleotides” Gene, 164:49-53; Stemmer (1995) “The Evolution of Molecular Computation” Science 270: 1510; Stemmer (1995) “Searching Sequence Space” Bio/Technology 13:549-553; Stemmer (1994) “Rapid evolution of a protein in vitro by DNA shuffling” Nature 370:389-391; and Stemmer (1994) “DNA shuffling by random fragmentation and reassembly: In vitro recombination for molecular evolution.” Proc. Natl. Acad. Sci. USA 91:10747-10751.

Mutational methods of generating diversity include, for example, site-directed mutagenesis (Ling et al. (1997) “Approaches to DNA mutagenesis: an overview” Anal Biochem. 254(2): 157-178; Dale et al. (1996) “Oligonucleotide-directed random mutagenesis using the phosphorothioate method” Methods Mol. Biol. 57:369-374; Smith (1985) “In vitro mutagenesis” Ann. Rev. Genet. 19:423-462; Botstein & Shortle (1985) “Strategies and applications of in vitro mutagenesis” Science 229:1193-1201; Carter (1986) “Site-directed mutagenesis” Biochem. J. 237:1-7; and Kunkel (1987) “The efficiency of oligonucleotide directed mutagenesis” in Nucleic Acids & Molecular Biology (Eckstein, F. and Lilley, D.M.J. eds., Springer Verlag, Berlin)); mutagenesis using uracil containing templates (Kunkel (1985) “Rapid and efficient site-specific mutagenesis without phenotypic selection” Proc. Natl. Acad. Sci. USA 82:488-492; Kunkel et al. (1987) “Rapid and efficient site-specific mutagenesis without phenotypic selection” Methods in Enzymol. 154, 367-382; and Bass et al. (1988) “Mutant Trp repressors with new DNA-binding specificities” Science 242:240-245); oligonucleotide-directed mutagenesis (Methods in Enzymol. 100: 468-500 (1983); Methods in Enzymol, 154: 329-350 (1987); Zoller & Smith (1982) “Oligonucleotide-directed mutagenesis using M13-derived vectors: an efficient and general procedure for the production of point mutations in any DNA fragment” Nucleic Acids Res. 10:6487-6500; Zoller & Smith (1983) “Oligonucleotide-directed mutagenesis of DNA fragments cloned into M13 vectors” Methods in Enzymol. 100:468-500; and Zoller & Smith (1987) “Oligonucleotide-directed mutagenesis: a simple method using two oligonucleotide primers and a single-stranded DNA template” Methods in Enzymol. 154:329-350); phosphorothioate-modified DNA mutagenesis (Taylor et al. (1985) “The use of phosphorothioate-modified DNA in restriction enzyme reactions to prepare nicked DNA” Nucl. Acids Res. 13: 8749-8764; Taylor et al. (1985) “The rapid generation of oligonucleotide-directed mutations at high frequency using phosphorothioate-modified DNA” Nucl. Acids Res. 13: 8765-8787 (1985); Nakamaye & Eckstein (1986) “Inhibition of restriction endonuclease Nci I cleavage by phosphorothioate groups and its application to oligonucleotide-directed mutagenesis” Nucl. Acids Res. 14: 9679-9698; Sayers et al. (1988) “Y-T Exonucleases in phosphorothioate-based oligonucleotide-directed mutagenesis” Nucl. Acids Res. 16:791-802; and Sayers et al. (1988) “Strand specific cleavage of phosphorothioate-containing DNA by reaction with restriction endonucleases in the presence of ethidium bromide” Nucl. Acids Res. 16: 803-814); mutagenesis using gapped duplex DNA (Kramer et al. (1984) “The gapped duplex DNA approach to oligonucleotide-directed mutation construction” Nucl. Acids Res. 12: 9441-9456; Kramer & Fritz (1987) Methods in Enzymol. “Oligonucleotide-directed construction of mutations via gapped duplex DNA” 154:350-367; Kramer et al. (1988) “Improved enzymatic in vitro reactions in the gapped duplex DNA approach to oligonucleotide-directed construction of mutations” Nucl. Acids Res. 16: 7207; and Fritz et al. (1988) “Oligonucleotide-directed construction of mutations: a gapped duplex DNA procedure without enzymatic reactions in vitro” Nucl. Acids Res. 16: 6987-6999).

Additional suitable methods include point mismatch repair (Kramer et al. (1984) “Point Mismatch Repair” Cell 38:879-887), mutagenesis using repair-deficient host strains (Carter et al. (1985) “Improved oligonucleotide site-directed mutagenesis using M13 vectors” Nucl. Acids Res. 13: 4431-4443; and Carter (1987) “Improved oligonucleotide-directed mutagenesis using M13 vectors” Methods in Enzymol. 154: 382-403), deletion mutagenesis (Eghtedarzadeh & Henikoff (1986) “Use of oligonucleotides to generate large deletions” Nucl. Acids Res. 14: 5115), restriction-selection and restriction-selection and restriction-purification (Wells et al. (1986) “Importance of hydrogen-bond formation in stabilizing the transition state of subtilisin” Phil. Trans. R. Soc. Lond. A 317: 415-423), mutagenesis by total gene synthesis (Nambiar et al (1984) “Total synthesis and cloning of a gene coding for the ribonuclease S protein” Science 223: 1299-1301; Sakamar and Khorana (1988) “Total synthesis and expression of a gene for the a-subunit of bovine rod outer segment guanine nucleotide-binding protein (transducin)” Nucl. Acids Res. 14: 6361-6372; Wells et al. (1985) “Cassette mutagenesis: an efficient method for generation of multiple mutations at defined sites” Gene 34:315-323; and Grundström et al. (1985) “Oligonucleotide-directed mutagenesis by microscale ‘shot-gun’ gene synthesis” Nucl. Acids Res. 13: 3305-3316), double-strand break repair (Mandecki (1986); Arnold (1993) “Protein engineering for unusual environments” Current Opinion in Biotechnology 4:450-455. “Oligonucleotide-directed double-strand break repair in plasmids of Escherichia coli: a method for site-specific mutagenesis” Proc. Natl. Acad. Sci. USA, 83:7177-7181). Additional details on many of the above methods can be found in Methods in Enzymology Volume 154, which also describes useful controls for trouble-shooting problems with various mutagenesis methods.

Additional details regarding various diversity generating methods can be found in the following U.S. patents, PCT publications, and EPO publications: U.S. Pat. No. 5,605,793 to Stemmer (Feb. 25, 1997), “Methods for In Vitro Recombination;” U.S. Pat. No. 5,811,238 to Stemmer et al. (Sep. 22, 1998) “Methods for Generating Polynucleotides having Desired Characteristics by Iterative Selection and Recombination;” U.S. Pat. No. 5,830,721 to Stemmer et al. (Nov. 3, 1998), “DNA Mutagenesis by Random Fragmentation and Reassembly;” U.S. Pat. No. 5,834,252 to Stemmer, et al. (Nov. 10, 1998) “End-Complementary Polymerase Reaction;” U.S. Pat. No. 5,837,458 to Minshull, et al. (Nov. 17, 1998), “Methods and Compositions for Cellular and Metabolic Engineering;” WO 95/22625, Stemmer and Crameri, “Mutagenesis by Random Fragmentation and Reassembly;” WO 96/33207 by Stemmer and Lipschutz “End Complementary Polymerase Chain Reaction;” WO 97/20078 by Stemmer and Crameri “Methods for Generating Polynucleotides having Desired Characteristics by Iterative Selection and Recombination;” WO 97/35966 by Minshull and Stemmer, “Methods and Compositions for Cellular and Metabolic Engineering;” WO 99/41402 by Punnonen et al. “Targeting of Genetic Vaccine Vectors;” WO 99/41383 by Punnonen et al. “Antigen Library Immunization;” WO 99/41369 by Punnonen et al. “Genetic Vaccine Vector Engineering;” WO 99/41368 by Punnonen et al. “Optimization of Immunomodulatory Properties of Genetic Vaccines;” EP 752008 by Stemmer and Crameri, “DNA Mutagenesis by Random Fragmentation and Reassembly;” EP 0932670 by Stemmer “Evolving Cellular DNA Uptake by Recursive Sequence Recombination;” WO 99/23107 by Stemmer et al, “Modification of Virus Tropism and Host Range by Viral Genome Shuffling;” WO 99/21979 by Apt et al., “Human Papillomavirus Vectors ;” WO 98/3183 7 by del Cardayre et a l. “Evolution of Whole Cells and Organisms by Recursive Sequence Recombination;” WO 98/27230 by Patten and Stemmer, “Methods and Compositions for Polypeptide Engineering;” WO 98/13487 by Stemmer et al., “Methods for Optimization of Gene Therapy by Recursive Sequence Shuffling and Selection,” WO 00/00632, “Methods for Generating Highly Diverse Libraries,” WO 00/09679, “Methods for Obtaining in Vitro Recombined Polynucleotide Sequence Banks and Resulting Sequences,” WO 98/42832 by Arnold et al., “Recombination of Polynucleotide Sequences Using Random or Defined Primers,” WO 99/29902 by Arnold et al., “Method for Creating Polynucleotide and Polypeptide Sequences,” WO 98/41653 by Vind, “An in Vitro Method for Construction of a DNA Library,” WO 98/41622 by Borchert et al., “Method for Constructing a Library Using DNA Shuffling,” and WO 98/42727 by Pati and Zarling, “Sequence Alterations using Homologous Recombination.”

Certain U.S. applications provide additional details regarding various diversity generating methods, including “Shuffling of Codon Altered Genes” by Patten et al. filed Sep. 28, 1999, (U.S. Ser. No. 09/407,800); “Evolution of Whole Cells and Organisms by Recursive Sequence Recombination”, by del Cardayre et al. filed Jul. 15, 1998 (U.S. Ser. No. 09/166,188), and Jul. 15, 1999 (USSN 09/354,922); “Oligonucleotide Mediated Nucleic Acid Recombination” by Crameri et al., filed Sep. 28, 1999 (U.S. Ser. No. 09/408,392), and “Oligonucleotide Mediated Nucleic Acid Recombination” by Crameri et al., filed Jan. 18, 2000 (PCT/US00/01203); “Use of Codon-Based Oligonucleotide Synthesis for Synthetic Shuffling” by Welch et al., filed Sep. 28, 1999 (U.S. Ser. No. 09/408,393); “Methods for Making Character Strings, Polynucleotides & Polypeptides Having Desired Characteristics” by Selifonov et al., filed Jan. 18, 2000, (PCT/US00/01202) and, e.g., “Methods for Making Character Strings, Polynucleotides & Polypeptides Having Desired Characteristics” by Selifonov et al., filed Jul. 18, 2000 (U.S. Ser. No. 09/618,579); “Methods of Populating Data Structures for Use in Evolutionary Simulations” by Selifonov and Stemmer (U.S. Ser. No. PCT/US00/01 138), filed Jan. 18, 2000; and “Single-Stranded Nucleic Acid Template-Mediated Recombination and Nucleic Acid Fragment Isolation” by Affholter, U.S. Ser. No. 60/186,482, filed Mar. 2, 2000.

In brief, several different general classes of sequence modification methods, such as mutation, recombination, etc. are applicable to the present invention and set forth, e.g., in the references above. That is the lipid acyl hydrolase nucleic acids of the invention can be generated from wild type sequences. Moreover, the lipid acyl hydrolase nucleic acid sequences of the invention can be modified to create modified sequences with the same or different activity.

The following exemplify some of the different types of preferred formats for diversity generation in the context of the present invention, including, e.g., certain recombination based diversity generation formats.

Nucleic acids can be recombined in vitro by any of a variety of techniques discussed in the references above, including e.g., DNAse digestion of nucleic acids to be recombined followed by ligation and/or PCR reassembly of the nucleic acids. For example, sexual PCR mutagenesis can be used in which random (or pseudo random, or even non-random) fragmentation of the DNA molecule is followed by recombination, based on sequence similarity, between DNA molecules with different but related DNA sequences, in vitro, followed by fixation of the crossover by extension in a polymerase chain reaction. This process and many process variants is described in several of the references above, e.g., in Stemmer (1994) Proc. Natl. Acad. Sci. USA 91:10747-10751. Thus, for example, nucleic acids encoding lipid acyl hydrolase with modified activity can be generated.

Similarly, nucleic acids can be recursively recombined in vivo, e.g., by allowing recombination to occur between nucleic acids in cells. Many such in vivo recombination formats are set forth in the references noted above. Such formats optionally provide direct recombination between nucleic acids of interest, or provide recombination between vectors, viruses, plasmids, etc., comprising the nucleic acids of interest, as well as other formats. Details regarding such procedures are found in the references noted above.

Whole genome recombination methods can also be used in which whole genomes of cells or other organisms are recombined, optionally including spiking of the genomic recombination mixtures with desired library components (e.g., genes corresponding to the pathways of the present invention). These methods have many applications, including those in which the identity of a target gene is not known. Details on such methods are found, e.g., in WO 98/31837 by del Cardayre et al. “Evolution of Whole Cells and Organisms by Recursive Sequence Recombination;” and in, e.g., PCT/US99/15972 by del Cardayre et al., also entitled “Evolution of Whole Cells and Organisms by Recursive Sequence Recombination.”

Synthetic recombination methods can also be used, in which oligonucleotides corresponding to targets of interest are synthesized and reassembled in PCR or ligation reactions which include oligonucleotides which correspond to more than one parental nucleic acid, thereby generating new recombined nucleic acids. Oligonucleotides can be made by standard nucleotide addition methods, or can be made, e.g., by tri-nucleotide synthetic approaches. Details regarding such approaches are found in the references noted above, including, e.g., “Oligonucleotide Mediated Nucleic Acid Recombination” by Crameri et al., filed Sep. 28, 1999 (U.S. Ser. No. 09/408,392), and “Oligonucleotide Mediated Nucleic Acid Recombination” by Crameri et al., filed Jan. 18, 2000 (PCT/US00/01203); “Use of Codon-Based Oligonucleotide Synthesis for Synthetic Shuffling” by Welch et al., filed Sep. 28, 1999 (U.S. Ser. No. 09/408,393); “Methods for Making Character Strings, Polynucleotides & Polypeptides Having Desired Characteristics” by Selifonov et al. , filed Jan. 18, 2000, (PCT/US00/01202); “Methods of Populating Data Structures for Use in Evolutionary Simulations” by Selifonov and Stemmer (PCT/US00/01 138), filed Jan. 18, 2000; and, e.g., “Methods for Making Character Strings, Polynucleotides & Polypeptides Having Desired Characteristics” by Selifonov et al., filed Jul. 18, 2000 (U.S. Ser. No. 09/618,579).

In silico methods of recombination can be effected in which genetic algorithms are used in a computer to recombine sequence strings which correspond to homologous (or even non-homologous) nucleic acids. The resulting recombined sequence strings are optionally converted into nucleic acids by synthesis of nucleic acids which correspond to the recombined sequences, e.g., in concert with oligonucleotide synthesis/gene reassembly techniques. This approach can generate random, partially random or designed variants. Many details regarding in silico recombination, including the use of genetic algorithms, genetic operators and the like in computer systems, combined with generation of corresponding nucleic acids (and/or proteins), as well as combinations of designed nucleic acids and/or proteins (e.g., based on cross-over site selection) as well as designed, pseudo-random or random recombination methods are described in “Methods for Making Character Strings, Polynucleotides & Polypeptides Having Desired Characteristics” by Selifonov et al., filed Jan. 18, 2000, (PCT/US00/01202) “Methods of Populating Data Structures for Use in Evolutionary Simulations” by Selifonov and Stemmer (PCT/US00/01138), filed Jan. 18, 2000; and, e.g., “Methods for Making Character Strings, Polynucleotides & Polypeptides Having Desired Characteristics” by Selifonov et al., filed Jul. 18, 2000 (U.S. Ser. No. 09/618,579). Extensive details regarding in silico recombination methods are found in these applications. This methodology is generally applicable to the present invention in providing for recombination of the lipid acyl hydrolase nucleic acids in silico and/or the generation of corresponding nucleic acids or proteins.

Many methods of accessing natural diversity, e.g., by hybridization of diverse nucleic acids or nucleic acid fragments to single-stranded templates, followed by polymerization and/or ligation to regenerate full-length sequences, optionally followed by degradation of the templates and recovery of the resulting modified nucleic acids can be similarly used. In one method employing a single-stranded template, the fragment population derived from the genomic library(ies) is annealed with partial, or, often approximately full length ssDNA or RNA corresponding to the opposite strand. Assembly of complex chimeric genes from this population is then mediated by nuclease-base removal of non-hybridizing fragment ends, polymerization to fill gaps between such fragments and subsequent single stranded ligation. The parental polynucleotide strand can be removed by digestion (e.g., if RNA or uracil-containing), magnetic separation under denaturing conditions (if labeled in a manner conducive to such separation) and other available separation/purification methods. Alternatively, the parental strand is optionally co-purified with the chimeric strands and removed during subsequent screening and processing steps. Additional details regarding this approach are found, e.g., in “Single-Stranded Nucleic Acid Template-Mediated Recombination and Nucleic Acid Fragment Isolation” by Affholter, U.S. Ser. No. 60/186,482, filed Mar. 2, 2000.

In another approach, single-stranded molecules are converted to double-stranded DNA (dsDNA) and the dsDNA molecules are bound to a solid support by ligand-mediated binding. After separation of unbound DNA, the selected DNA molecules are released from the support and introduced into a suitable host cell to generate a library enriched sequences which hybridize to the probe. A library produced in this manner provides a desirable substrate for further diversification using any of the procedures described herein.

Any of the preceding general recombination formats can be practiced in a reiterative fashion (e.g., one or more cycles of mutation/recombination or other diversity generation methods, optionally followed by one or more selection methods) to generate a more diverse set of recombinant nucleic acids.

Mutagenesis employing polynucleotide chain termination methods have also been proposed (see e.g., U.S. Pat. No. 5,965,408, “Method of DNA reassembly by interrupting synthesis” to Short, and the references above), and can be applied to the present invention. In this approach, double stranded DNAs corresponding to one or more genes sharing regions of sequence similarity are combined and denatured, in the presence or absence of primers specific for the gene. The single stranded polynucleotides are then annealed and incubated in the presence of a polymerase and a chain terminating reagent (e.g., ultraviolet, gamma or X-ray irradiation; ethidium bromide or other intercalators; DNA binding proteins, such as single strand binding proteins, transcription activating factors, or histones; polycyclic aromatic hydrocarbons; trivalent chromium or a trivalent chromium salt; or abbreviated polymerization mediated by rapid thermocycling; and the like), resulting in the production of partial duplex molecules. The partial duplex molecules, e.g., containing partially extended chains, are then denatured and reannealed in subsequent rounds of replication or partial replication resulting in polynucleotides which share varying degrees of sequence similarity and which are diversified with respect to the starting population of DNA molecules. Optionally, the products, or partial pools of the products, can be amplified at one or more stages in the process. Polynucleotides produced by a chain termination method, such as described above, are suitable substrates for any other described recombination format.

Diversity also can be generated in nucleic acids or populations of nucleic acids using a recombinational procedure termed “incremental truncation for the creation of hybrid enzymes” (“ITCHY”) described in Osterneier et al. (1999) “A combinatorial approach to hybrid enzymes independent of DNA homology” Nature Biotech 17:1205. This approach can be used to generate an initial a library of variants which can optionally serve as a substrate for one or more in vitro or in vivo recombination methods. See, also, Ostermeier et al. (1999) “Combinatorial Protein Engineering by Incremental Truncation,” Proc. Natl. Acad. Sci. USA, 96: 3562-67; Ostermeier et al. (1999), “Incremental Truncation as a Strategy in the Engineering of Novel Biocatalysts,” Biological and Medicinal Chemistry, 7: 2139-44.

Mutational methods which result in the alteration of individual nucleotides or groups of contiguous or non-contiguous nucleotides can be favorably employed to introduce nucleotide diversity. Thus, modified lipid acyl hydrolase nucleic acids of the invention can be generated, including for optimized codon usage for an organism of interest, as well as nucleic acids encoding lipid acyl hydrolase polypeptides with improved and/or modified activity. Many mutagenesis methods are found in the above-cited references; additional details regarding mutagenesis methods can be found in following, which can also be applied to the present invention.

For example, error-prone PCR can be used to generate nucleic acid variants. Using this technique, PCR is performed under conditions where the copying fidelity of the DNA polymerase is low, such that a high rate of point mutations is obtained along the entire length of the PCR product. Examples of such techniques are found in the references above and, e.g., in Leung et al. (1989) Technique 1: 11-15 and Caldwell et al. (1992) PCR Methods Applic. 2:28-33. Similarly, assembly PCR can be used, in a process which involves the assembly of a PCR product from a mixture of small DNA fragments. A large number of different PCR reactions can occur in parallel in the same reaction mixture, with the products of one reaction priming the products of another reaction.

Oligonucleotide directed mutagenesis can be used to introduce site-specific mutations in a nucleic acid sequence of interest. Examples of such techniques are found in the references above and, e.g., in Reidhaar-Olson et al. (1988) Science, 241:53-57. Similarly, cassette mutagenesis can be used in a process that replaces a small region of a double stranded DNA molecule with a synthetic oligonucleotide cassette that differs from the native sequence. The oligonucleotide can contain, e.g., completely and/or partially randomized native sequence(s).

Recursive ensemble mutagenesis is a process in which an algorithm for protein mutagenesis is used to produce diverse populations of phenotypically related mutants, members of which differ in amino acid sequence. This method uses a feedback mechanism to monitor successive rounds of combinatorial cassette mutagenesis. Examples of this approach are found in Arkin & Youvan (1992) Proc. Natl. Acad. Sci. USA 89:7811-7815.

Exponential ensemble mutagenesis can be used for generating combinatorial libraries with a high percentage of unique and functional mutants. Small groups of residues in a sequence of interest are randomized in parallel to identify, at each altered position, amino acids which lead to functional proteins. Examples of such procedures are found in Delegrave & Youvan (1993) Biotechnology Research 11:1548-1552.

In vivo mutagenesis can be used to generate random mutations in any cloned DNA of interest by propagating the DNA, e.g., in a strain of E. coil that carries mutations in one or more of the DNA repair pathways. These “mutator” strains have a higher random mutation rate than that of a wild-type parent. Propagating the DNA in one of these strains will eventually generate random mutations within the DNA. Such procedures are described in the references noted above.

Other procedures for introducing diversity into a genome, e.g. a bacterial, fungal, animal or plant genome can be used in conjunction with the above described and/or referenced methods. For example, in addition to the methods above, techniques have been proposed which produce nucleic acid multimers suitable for transformation into a variety of species (see, e.g., Schellenberger U.S. Pat. No. 5,756,316 and the references above). Transformation of a suitable host with such multimers, consisting of genes that are divergent with respect to one another, (e.g., derived from natural diversity or through application of site directed mutagenesis, error prone PCR, passage through mutagenic bacterial strains, and the like), provides a source of nucleic acid diversity for DNA diversification, e.g., by an in vivo recombination process as indicated above.

Alternatively, a multiplicity of monomeric polynucleotides sharing regions of partial sequence similarity can be transformed into a host species and recombined in vivo by the host cell. Subsequent rounds of cell division can be used to generate libraries, members of which, include a single, homogenous population, or pool of monomeric polynucleotides. Alternatively, the monomeric nucleic acid can be recovered by standard techniques, e.g., PCR and/or cloning, and recombined in any of the recombination formats, including recursive recombination formats, described above.

Methods for generating multispecies expression libraries have been described (in addition to the reference noted above, see, e.g., Peterson et al. (1998) U.S. Pat. No. 5,783,431 “Methods for Generating and Screening Novel Metabolic Pathways,” and Thompson, et al. (1998) U.S. Pat. No. 5,824,485 Methods for Generating and Screening Novel Metabolic Pathways) and their use to identify protein activities of interest has been proposed (In addition to the references noted above, see, Short (1999) U.S. Pat. No. 5,958,672 “Protein Activity Screening of Clones Having DNA from Uncultivated Microorganisms”). Multispecies expression libraries include, in general, libraries comprising cDNA or genomic sequences from a plurality of species or strains, operably linked to appropriate regulatory sequences, in an expression cassette. The eDNA and/or genomic sequences are optionally randomly ligated to further enhance diversity. The vector can be a shuttle vector suitable for transformation and expression in more than one species of host organism, e.g., bacterial species, eukaryotic cells. In some cases, the library is biased by preselecting sequences which encode a protein of interest, or which hybridize to a nucleic acid of interest. Any such libraries can be provided as substrates for any of the methods herein described.

The above described procedures have been largely directed to increasing nucleic acid and/ or encoded protein diversity. However, in many cases, not all of the diversity is useful, e.g., functional, and contributes merely to increasing the background of variants that must be screened or selected to identify the few favorable variants. In some applications, it is desirable to preselect or prescreen libraries (e.g., an amplified library, a genomic library, a cDNA library, a normalized library, etc.) or other substrate nucleic acids prior to diversification, e.g., by recombination-based mutagenesis procedures, or to otherwise bias the substrates towards nucleic acids that encode functional products. For example, in the case of antibody engineering, it is possible to bias the diversity generating process toward antibodies with functional antigen binding sites by taking advantage of in vivo recombination events prior to manipulation by any of the described methods. For example, recombined CDRs derived from B cell cDNA libraries can be amplified and assembled into framework regions (e.g., Jirholt et al. (1998) “Exploiting sequence space: shuffling in vivo formed complementarity determining regions into a master framework” Gene 215: 471) prior to diversifying according to any of the methods described herein.

Libraries can be biased towards nucleic acids which encode proteins with desirable enzyme activities. For example, after identifying a clone from a library which exhibits a specified activity, the clone can be mutagenized using any known method for introducing DNA alterations. A library comprising the mutagenized homologues is then screened for a desired activity, which can be the same as or different from the initially specified activity. An example of such a procedure is proposed in Short (1999) U.S. Pat. No. 5,939,250 for “Production of Enzymes Having Desired Activities by Mutagenesis.” Desired activities can be identified by any method known in the art. For example, WO 99/10539 proposes that gene libraries can be screened by combining extracts from the gene library with components obtained from metabolically rich cells and identifying combinations which exhibit the desired activity. It has also been proposed (e.g., WO 98/58085) that clones with desired activities can be identified by inserting bioactive substrates into samples of the library, and detecting bioactive fluorescence corresponding to the product of a desired activity using a fluorescent analyzer, e.g., a flow cytometry device, a CCD, a fluorometer, or a spectrophotometer.

Libraries can also be biased towards nucleic acids which have specified characteristics, e.g., hybridization to a selected nucleic acid probe. For example, application WO 99/10539 proposes that polynucleotides encoding a desired activity (e.g., an enzymatic activity, for example: a lipase, an esterase, a protease, a glycosidase, a glycosyl transferase, a phosphatase, a kinase, an oxygenase, a peroxidase, a hydrolase, a hydratase, a nitrilase, a transaminase, an amidase or an acylase) can be identified from among genomic DNA sequences in the following manner. Single stranded DNA molecules from a population of genomic DNA are hybridized to a ligand-conjugated probe. The genomic DNA can be derived from either a cultivated or uncultivated microorganism, or from an environmental sample. Alternatively, the genomic DNA can be derived from a multicellular organism, or a tissue derived therefrom. Second strand orsynthesis can be conducted directly from the hybridization probe used in the capture, with or without prior release from the capture medium or by a wide variety of other strategies known in the art. Alternatively, the isolated single-stranded genomic DNA population can be fragmented without further cloning and used directly in, e.g., a recombination-based approach, that employs a single-stranded template, as described above.

“Non-Stochastic” methods of generating nucleic acids and polypeptides are alleged in Short “Non-Stochastic Generation of Genetic Vaccines and Enzymes” WO 00/46344. These methods, including proposed non-stochastic polynucleotide reassembly and site-saturation mutagenesis methods can be applied to the present invention as well.

It will readily be appreciated that any of the above described techniques suitable for enriching a library prior to diversification can also be used to screen the products, or libraries of products, produced by the diversity generating methods.

Kits for mutagenesis, library construction and other diversity generation methods are also commercially available. For example, kits are available from, e.g., Stratagene (e.g., QuickChange™ site-directed mutagenesis kit; and Chameleon™ double-stranded, site-directed mutagenesis kit), Bio/Can Scientific, Bio-Rad (e.g., using the Kunkel method described above), Boehringer Mannheim Corp., Clonetech Laboratories, DNA Technologies, Epicentre Technologies (e.g., 5 prime 3 prime kit); Genpak Inc, Lemargo Inc, Life Technologies (Gibco BRL), New England Biolabs, Pharmacia Biotech, Promega Corp., Quantum Biotechnologies, Amersham International plc (e.g., using the Eckstein method above), and Anglian Biotechnology Ltd (e.g., using the Carter/Winter method above).

The above references provide many mutational formats, including recombination, recursive recombination, recursive mutation and combinations or recombination with other forms of mutagenesis, as well as many modifications of these formats. Regardless of the diversity generation format that is used, the nucleic acids of the invention can be recombined (with each other, or with related (or even unrelated) sequences) to produce a diverse set of recombinant nucleic acids, including, e.g., sets of homologous nucleic acids, as well as corresponding polypeptides.

Modification of lipid acyl hydrolase nucleic acids for common codon usage in an organism

The polynucleotide sequence encoding a particular lipid acyl hydrolase can be altered to coincide with the codon usage of a particular host. For example, the codon usage of a monocot plant can be used to derive a polynucleotide that encodes a lipid acyl hydrolase polypeptide of the invention and comprises preferred monocot codons. The frequency of preferred codon usage exhibited by a host cell can be calculated by averaging frequency of preferred codon usage in a large number of genes expressed by the host cell. This analysis is preferably limited to genes that are highly expressed by the host cell. U.S. Pat. No. 5,824,864, for example, provides the frequency of codon usage by highly expressed genes exhibited by dicotyledonous plants and monocotyledonous plants.

When synthesizing a gene for improved expression in a host cell, it is desirable to design the gene such that its frequency of codon usage approaches the frequency of preferred codon usage of the host cell. The percent deviation of the frequency of preferred codon usage for a synthetic gene from that employed by a host cell is calculated first by determining the percent deviation of the frequency of usage of a single codon from that of the host cell followed by obtaining the average deviation over all codons.

Screens for determining insecticidal activity of lipid acyl hydrolases

To test the effect of the modified acyl hydrolases for insecticidal activity, one may perform a bioassay of these insects. General methods to perform such assays are known in the art (see, e.g., PCT WO 98/54327). In general, to test for the effect of proteins, one adds the sample to be tested to the food that the insects consume. The protein sample may be purified protein, or it may be a crude lysate, bacterial or eucaryotic cell, homogenized plant tissue, or culture supernatant containing the protein of interest. Usually such tests are performed in small dishes or in multi-well plates. Artificial diets that allow the rearing and/or bioassay of many insect species are commercially available, for example from Bioserv, Inc. Alternatively, one can perform such assays using leaf discs or whole leaves. Often, the protein sample is added to the surface of the insect diet or leaf, then allowed to dry. Alternatively, one can incorporate the protein sample directly into the molten insect diet, before dispensing into the dish or well. After the samples have dried, one or more larvae are added to each well. Alternatively, one may place eggs in the well, which hatch in 12-36 hours to yield larvae. The larvae feed upon the leaf or diet, and consequently ingest the protein to be tested. After one or several days, the insects are observed to note mortality, stunting of growth, or other physiological responses. One may perform this test with many replicates, and/or with different dilutions of the proteins. After noting mortality on several dilutions of toxin, one may determine LC₅₀ and/or other measures of mortality by graphing the mortality vs. dose, or by using statistical treatments such as probit analysis. One may quantify the amount of stunting or other more subtle effects on such insects by weighing the insects after the incubation time, and comparing the weights obtained with those observed in a control assay where the insects were not exposed to toxin. After such an assay, acyl hydrolases causing increased mortality or stunting of the insects can be identified.

In some embodiments, the polypeptides exhibit an LC50 of approximately 10 μg/ml, more preferably less than 5 μg/ml, and more preferably 1 μg/ml against insects such as corn rootworm.

Isolation of lipid acyl hydrolase nucleic acids

Generally, the nomenclature and the laboratory procedures in recombinant DNA technology described below are those well known and commonly employed in the art. Standard techniques are used for cloning, DNA and RNA isolation, amplification and purification. Generally enzymatic reactions involving DNA ligase, DNA polymerase, restriction endonucleases and the like are performed according to the manufacturer's specifications. These techniques and various other techniques are generally performed according to Sambrook et al., Molecular Cloning - A Laboratory Manual, Cold Spring Harbor Laboratory, Cold Spring Harbor, New York, (1989) or Current Protocols in Molecular Biology Volumes 1-3, John Wiley & Sons, Inc. (1994-1998) (“Ausubel et al.”).

The isolation of lipid acyl hydrolase nucleic acids may be accomplished by a number of techniques. For instance, oligonucleotide probes based on the sequences disclosed here can be used to identify the desired gene in a cDNA or genomic DNA library. To construct genomic libraries, large segments of genomic DNA are generated by random fragmentation, e.g. using restriction endonucleases, and are ligated with vector DNA to form concatemers that can be packaged into the appropriate vector. To prepare a cDNA library, mRNA is isolated from the desired organ, such as leaves, and a cDNA library which contains a lipid acyl hydrolase gene transcript is prepared from the mRNA. Alternatively, cDNA may be prepared from mRNA extracted from other tissues in which lipid acyl hydrolase genes or homologues are expressed.

The cDNA or genomic library can then be screened using a probe based upon the sequence of a cloned lipid acyl hydrolase gene disclosed here. Probes may be used to hybridize with genomic DNA or cDNA sequences to isolate homologous genes in the same or different plant species. Alternatively, antibodies raised against a lipid acyl hydrolase polypeptide can be used to screen an mRNA expression library.

Alternatively, the nucleic acids of interest can be amplified from nucleic acid samples using amplification techniques. For instance, polymerase chain reaction (PCR) technology can be used to amplify the sequences of lipid acyl hydrolase genes directly from genomic DNA, from cDNA, from genomic libraries or cDNA libraries. PCR and other in vitro amplification methods may also be useful, for example, to clone nucleic acid sequences that code for proteins to be expressed, to make nucleic acids to use as probes for detecting the presence of the desired mRNA in samples, for nucleic acid sequencing, or for other purposes. For a general overview of PCR, see PCR Protocols: A Guide to Methods and Applications. (Innis, M, Gelfand, D., Sninsky, J. and White, T., eds.), Academic Press, San Diego (1990). Appropriate primers and probes for identifying lipid acyl hydrolase sequences from plant tissues are generated from comparisons of the sequences provided here (e.g. SEQ ID NO:1, SEQ ID NO:3, etc.).

Polynucleotides may also be synthesized by well-known techniques as described in the technical literature. See, e.g., Carruthers et al., Cold Spring Harbor Symp. Quant. Biol. 47:411-418 (1982), and Adams et al., J. Am. Chem. Soc. 105:661 (1983). Double stranded DNA fragments may then be obtained either by synthesizing the complementary strand and annealing the strands together under appropriate conditions, or by adding the complementary strand using DNA polymerase with an appropriate primer sequence.

One useful method to produce the nucleic acids of the invention is to isolate and modify the wild type pentin polynucleotide sequence, (e.g., SEQ ID NO:43). See, also, PCT No. WO 98/54327. Several methods for sequence-specific mutagenesis of a nucleic acid are known and are described above. In addition, Ausubel et al., supra, describes oligonucleotide-directed mutagenesis as well as directed mutagenesis of nucleic acids using PCR. Such methods are useful to insert specific codon changes in the nucleic acids of the invention into the wild type pentin polynucleotide.

Preparation of recombinant vectors

Typical vectors contain transcription and translation terminators, transcription and translation initiation sequences, and promoters useful for regulation of the expression of the particular nucleic acid. The vectors optionally comprise generic expression cassettes containing at least one independent terminator sequence, sequences permitting replication of the cassette in eukaryotes, or prokaryotes, or both, (e.g., shuttle vectors) and selection markers for both prokaryotic and eukaryotic systems. Vectors are suitable for replication and integration in prokaryotes, eukaryotes, or preferably both. See, Giliman & Smith, Gene 8:81 (1979); Roberts, et al., Nature, 328:731 (1987); Schneider, B., et al., Protein Expr. Purif. 6435:10 (1995); Berger, Sambrook, Ausubel (all supra). A catalogue of Bacteria and Bacteriophages useful for cloning is provided, e.g., by the ATCC, e.g., The ATCC Catalogue of Bacteria and Bacteriophage (1992) Gherna et al. (eds) published by the ATCC. Additional basic procedures for sequencing, cloning and other aspects of molecular biology and underlying theoretical considerations are also found in Watson et al. (1992) Recombinant DNA Second Edition Scientific American Books, NY.

To use isolated sequences in the above techniques, recombinant DNA vectors suitable for transformation of plant cells are prepared. Techniques for transforming a wide variety of higher plant species are well known and described in the technical and scientific literature. See, for example, Weising et al. Ann. Rev. Genet. 22:421-477 (1988). A DNA sequence coding for the desired polypeptide, for example a cDNA sequence encoding a full length protein, will preferably be combined with transcriptional and translational initiation regulatory sequences which will direct the transcription of the sequence from the gene in the intended tissues of the transformed plant. Native or heterologous promoters can be operatively linked to transcriptional sequences.

For example, for overexpression, a plant promoter fragment may be employed which will direct expression of the gene in all tissues of a regenerated plant. Such promoters are referred to herein as “constitutive” promoters and are active under most environmental conditions and states of development or cell differentiation. Examples of constitutive promoters include the cauliflower mosaic virus (CaMV) 35S transcription initiation region, the 1′- or 2′- promoter derived from T-DNA of Agrobacterium tumafaciens, and other transcription initiation regions from various plant genes known to those of skill. Such genes include for example, ACT11 from Arabidopsis (Huang et al. Plant Mol. Biol. 33:125-139 (1996)), Cat3 from Arabidopsis (GenBank No. U43147, Zhong et al., Mol. Gen. Genet. 251:196-203 (1996)), the gene encoding stearoyl-acyl carrier protein desaturase from Brassica napus (Genbank No. X74782, Solocombe et al. Plant Physiol. 104:1167-1176 (1994)), GPcl from maize (GenBank No. XI 5596, Martinez et al. J. Mol. Biol 208:551-565 (1989)), and Gpc2 from maize (GenBank No. U45855, Manjunath et al., Plant Mol. Biol. 33:97-112 (1997)).

Alternatively, the plant promoter may direct expression of lipid acyl hydrolase nucleic acids in a specific tissue, organ or cell type (i.e. tissue-preferred promoters) or may be otherwise under more precise environmental or developmental control (i.e. inducible promoters). Examples of environmental conditions that may effect transcription by inducible promoters include anaerobic conditions, elevated temperature, the presence of light, or sprayed with chemicals/hormones. Tissue-preferred promoters can be inducible. Similarly, tissue-preferred promoters may only promote transcription within a certain time frame of developmental stage within that tissue. Other tissue specific promoters may be active throughout the life cycle of a particular tissue. One of skill will recognize that a tissue-preferred promoter may drive expression of operably linked sequences in tissues other than the target tissue. Thus, as used herein, a tissue-preferred promoter is one that drives expression preferentially in the target tissue or cell type, but may also lead to some expression in other tissues as well.

A number of tissue-preferred promoters can also be used in the invention. With the appropriate promoter, any organ can be targeted, such as shoot vegetative organs/structures (e.g. leaves, stems and tubers), roots, flowers and floral organs/structures (e.g. bracts, sepals, petals, stamens, carpels, anthers and ovules), seed (including embryo, endosperm, and seed coat) and fruit. For instance, promoters that direct expression of nucleic acids in leaves, roots or flowers are useful for enhancing resistance to pests that infect those organs. For expression of a lipid acyl hydrolase polynucleotide in the aerial vegetative organs of a plant, photosynthetic organ-specific promoters, such as the RBCS promoter (Khoudi, et al., Gene 197:343, 1997), can be used. Root-specific expression of lipid acyl hydrolase polynucleotides can be achieved under the control of a root-specific promoter, e.g., from the ANR1 gene (Zhang & Forde, Science, 279:407, 1998). Other examples include Hirel, et al., Plant Molecular Biology 20(2):207-218 (1992), which describes a root-specific glutamine synthetase gene from soybean and Keller, et al., The Plant Cell 3(10):1051-1061 (1991), which describes a root-specific control element in the GRP 1.8 gene of French bean. Any strong, constitutive promoters, such as the CaMV 35S promoter, can be used for the expression of lipid acyl hydrolase polynucleotides throughout the plant.

If proper polypeptide expression is desired, a polyadenylation region at the 3′-end of the coding region should be included. The polyadenylation region can be derived from the natural gene, from a variety of other plant genes, or from T-DNA.

The vector comprising the sequences (e.g., promoters or coding regions) from genes of the invention will typically comprise a marker gene that confers a selectable phenotype on plant cells. For example, the marker may encode biocide resistance, particularly antibiotic resistance, such as resistance to kanamycin, G418, bleomycin, hygromycin, or herbicide resistance, such as resistance to chlorosulfuron or Basta.

Production of transgenic plants

DNA constructs of the invention may be introduced into the genome of the desired plant host by a variety of conventional techniques. For example, the DNA construct may be introduced directly into the genomic DNA of the plant cell using techniques such as electroporation and microinjection of plant cell protoplasts, or the DNA constructs can be introduced directly to plant tissue using ballistic methods, such as DNA particle bombardment.

Microinjection techniques are known in the art and well described in the scientific and patent literature. The introduction of DNA constructs using polyethylene glycol precipitation is described in Paszkowski et al. Embo. J. 3:2717-2722 (1984). Electroporation techniques are described in Fromm et al. Proc. Natl. Acad. Sci. USA 82:5824 (1985). Ballistic transformation techniques are described in Klein et al. Nature 327:70-73 (1987).

Alternatively, the DNA constructs may be combined with suitable T-DNA flanking regions and introduced into a conventional Agrobacterium tumefaciens host vector. The virulence functions of the Agrobacterium tumefaciens host will direct the insertion of the construct and adjacent marker into the plant cell DNA when the cell is infected by the bacteria. Agrobacterium tumefaciens-mediated transformation techniques, including disarming and use of binary vectors, are well described in the scientific literature. See, for example Horsch et al. Science 233:496-498 (1984), and Fraley et al. Proc. Natl. Acad. Sci. USA 80:4803 (1983) and Gene Transfer to Plants, Potrykus, ed. (Springer-Verlag, Berlin 1995).

Transformed plant cells which are derived by any of the above transformation techniques can be cultured to regenerate a whole plant which possesses the transformed genotype and thus the desired phenotype such as increased seed mass. Such regeneration techniques rely on manipulation of certain phytohormones in a tissue culture growth medium, typically relying on a biocide and/or herbicide marker that has been introduced together with the desired nucleotide sequences. Plant regeneration from cultured protoplasts is described in Evans et al., Protoplasts Isolation and Culture, Handbook of Plant Cell Culture, pp. 124-176, MacMillilan Publishing Company, New York, 1983; and Binding, Regeneration of Plants, Plant Protoplasts, pp. 21-73, CRC Press, Boca Raton, 1985. Regeneration can also be obtained from plant callus, explants, organs, or parts thereof. Such regeneration techniques are described generally in Klee et al. Ann. Rev. of Plant Phys. 38:467-486 (1987).

The nucleic acids of the invention can be used to confer desired traits on essentially any plant. Thus, the invention has use over a broad range of plants, including species from the genera Anacardium, Arachis, Asparagus, Atropa, Avena, Brassica, Citrus, Citrullus, Capsicum, Carthamus, Cocos, Coffea, Cucumis, Cucurbita, Daucus, Elaeis, Fragaria, Glycine, Gossypium, Helianthus, Heterocallis, Hordeum, Hyoscyamus, Lactuca, Linum, Lolium, Lupinus, Lycopersicon, Malus, Manihot, Majorana, Medicago, Nicotiana, Olea, Oryza, Panieum, Pannesetum, Persea, Phaseolus, Pistachia, Pisum, Pyrus, Prunus, Raphanus, Ricinus, Secale, Senecio, Sinapis, Solanum, Sorghum, Theobromus, Trigonella, Triticum, Vicia, Vitis, Vigna, and Zea. In one aspect of the invention, the methods and composition of the invention are applied to maize, potato, soybean or cotton plants.

One of skill will recognize that after the expression cassette is stably incorporated in transgenic plants and confirmed to be operable, it can be introduced into other plants by sexual crossing. Any of a number of standard breeding techniques can be used, depending upon the species to be crossed.

Using known procedures one of skill can screen for plants of the invention by detecting the increase or decrease of lipid acyl hydrolase mRNA or protein in transgenic plants. Methods for detecting and quantitation of mRNAs or proteins are well known in the art.

Methods of assaying lipid acyl hydrolase activity

A variety of assays can be used to determine whether a particular polypeptide has lipid acyl hydrolase activity. Typically, activity of an acid lipid hydrolase candidate is compared to a negative control (i.e., a sample that comprises no proteins with acid lipid hydrolase activity or that comprise the reagents of the sample but contain no other proteins). As an additional control, a candidate polypeptide's lipid acyl hydrolase activity can be compared to the pentin polypeptide (SEQ ID NO:41) to identify candidates with improved enzymatic activity.

A simple test includes assaying for the ability of a polypeptide to hydrolyze p-nitrophenyl caprylate. Typically a purified polypeptide, crude bacterial lysate, or crude plant lysate containing the candidate polypeptide is added to a buffered solution (e.g., 100 mM Tris pH8.2, 100 mM KCl) and then mixed with the p-nitrophenyl caprylate substrate in a buffer (e.g., 1% triton X-100, 50 mM KCl, 100 mM Tris pH8.2). A spectrophotometer can then be used to detect changes of absorbance at 410 nm, thereby assaying hydrolysis of the substrate.

One alternate screen involves growing microorganisms expressing the candidate polypeptides on a solid medium, and overlaying the microorganisms with an agarose solution containing N,N-dimethyl formamide, α-napthyl caproate, α-napthyl caprylate and α-napthyl caprate, and looking for formation of a brown precipitate indicating hydrolysis of the caproate, caprylate, or caprate.

Methods of enhancing plant resistance to insect pests

The present invention provides for method of enhancing plant resistance to pests such as insects by expressing lipid acyl hydrolase polynucleotides and/or polypeptides in plants. The insecticidal activity of certain lipid acid hydrolase proteins has been described previously. See, e.g., PCT WO 98/54327, U.S. Pat. Nos. 5,824,864, 5,882,668, and 5,743,477. For example, in preferred embodiments, the lipid acyl hydrolase polypeptides of the invention can be incorporated into the tissues of a susceptible plant so that in the course of infesting the plant, the insect consumes insect-controlling amounts of the selected lipid acyl hydrolase.

Enhanced resistance to any insect pest is contemplated, including pests such as corn rootworms, potato beetles, armyworms, borers, cutworms, wireworms, earworms and aphids. Other insect pests are described in, e.g., PCT WO98/54327 and Stoetzel, COMMON NAMES OF INSECTS & RELATED ORGANISMS (1989). Enhanced resistance is generally achieved by introducing into a plant, or tissue or cell thereof, a structural gene encoding a lipid acyl hydrolase of the invention, operably linked to plant regulatory sequences which cause expression of the lipid acyl hydrolase gene in the plant.

In some embodiments, the expression of high quantities of lipid acyl hydrolases may be deleterious to the plant itself. The use of a signal sequence to secrete or sequester the polypeptides in a selected organelle allows the polypeptides to be in a metabolically inert location until released in the gut environment of an insect pathogen. See, e.g., Von Heijne et al., Plant Mol. Biol. Rep. 9:104-126 (1991); Clark et al., J. Biol. Chem. 264:17544-17550 (1989); Shah et al., Science 233:478-481 (1986). Moreover, some proteins are accumulated to higher levels in transgenic plants when they are secreted from the cells, rather than stored in the cytosol (Hiatt, et al., Nature 342:76-78 (1989)).

As an alternative to expressing the polypeptides of the invention in plant cells, the presentation of the polypeptides can be made by formulating the polypeptide into an agricultural composition that is applied to the plant. Thus, presentation of the agricultural composition may be achieved by external application either directly or in the vicinity of the plants or plant parts. The agricultural compositions may be applied to the environment of the insect pest(s), e.g., plants, soil or water, by spraying, dusting, sprinkling, or the like.

The present invention further contemplates using recombinant hosts (e.g., microbial hosts and insect viruses) transformed with a gene encoding the lipid acyl hydrolase polypeptides of the invention and applied on or near a selected plant or plant part susceptible to attack by a target insect. The hosts may be capable of colonizing a plant tissue susceptible to insect infestation or of being applied as dead or non-viable cells containing the lipid acyl hydrolase. Microbial hosts of particular interest will be the prokaryotes and the lower eukaryotes, such as fungi.

Characteristics of microbial hosts for encapsulating a lipid acyl hydrolase include protective qualities for the polypeptide, such as thick cell walls, pigmentation, and intracellular packaging or formation of inclusion bodies; leaf affinity; lack of mammalian toxicity; attractiveness to pests for ingestion; ease of killing and fixing without damage to the plant non-specific lipid acyl hydrolase; and the ability to be treated to prolong the activity of the plant non-specific lipid acyl hydrolase. Characteristics of microbial hosts for colonizing a plant include non-phytotoxicity; ease of introducing a genetic sequence encoding a plant non-specific lipid acyl hydrolase, availability of expression systems, efficiency of expression and stability of the insecticide in the host.

Examples of prokaryotes, both Gram-negative and -positive, that are potentially useful for encapsulating lipid acyl hydrolases include Enterobacteriaceae, such as Escherichia; Bacillaceae; Rhizoboceae, such as Rhizobium and Rhizobacter; Spirillaceae (such as photobacterium), Zymomonas, Serratia, Aeromonas, Vibrio, Desulfovibrio, Spirillum; Lactobacillaceae; Pseudomonadaceae (such as Pseudomonas and Acetobacter); Azotobacteraceae and Nitrobacteraceae. Among eukaryotes are fungi (such as Phycomycetes and Ascomycetes), which includes yeast (such as Saccharomyces and Schizosaccharomyces); and Basidiomycetes yeast (such as Rhodotorula, Aureobasidium, Sporobolomyces) and the like.

The present invention also contemplates the use of a baculovirus containing a gene encoding a lipid acyl hydrolase polypeptide of the invention. Baculoviruses including those that infect Heliothis virescens (cotton bollworm), Orgyla pseudotsugata (Douglas fir tussock moth), Lymantria dispar (gypsy moth), Autographica californica (alfalfa looper), Neodiprion sertifer (European pine fly) and Laspeyresia pomonella (coddling moth) have been registered and used as pesticides (see U.S. Pat. No. 4,745,051 and EP 175 852).

The recombinant host may be formulated in a variety of ways. It may be employed in wettable powders, granules or dusts, or by mixing with various inert materials, such as inorganic minerals (phyllosilicates, carbonates, sulfates, phosphates, and the like) or botanical materials (powdered corncobs, rice hulls, walnut shells, and the like). The formulations may include spreader-sticker adjuvants, stabilizing agents, other insecticidal additives surfactants, and bacterial nutrients or other agents to enhance growth or stabilize bacterial cells. Liquid formulations may be aqueous-based or non-aqueous and employed as foams, gels, suspensions, emulsifiable concentrates, or the like. The ingredients may include Theological agents, surfactants, emulsifiers, dispersants, or polymers. In general, inoculants can be applied at any time during plant growth. Inoculation of large fields can be accomplished most effectively by spraying.

Selecting for plants with enhanced resistance

Plants with enhanced resistance can be selected in many ways known to those of skill in the art. For example, to assess resistance to insect attack, transgenic plants expressing the polypeptides of the invention are infested with an insect pest to which the wild type plant is susceptible. In some cases, for instance, the soil is infested with insect eggs. The plants are then monitored over multiple weeks (e.g., four weeks) for, e.g., viability, height, root mass and leaf area.

Combining lipid acyl hydrolase polypeptides of the invention with other proteins to enhance pest resistance

The polypeptides of the invention may be used alone or in combination with other proteins or agents to control different insect pests. Other insecticidal proteins include those from Bacillus, including (Bt) δ-endotoxins and vegetative insecticidal proteins, as well as protease inhibitors (both serine and cysteine types), lectins, α-amylases, peroxidases, cholesterol oxidase, and the like.

### EXAMPLES

The following examples are offered to illustrate, but not to limit the claimed invention.

### Example 1

### Library of genes related to the wild type Pentin cDNA clone

A library of nucleic acids related to the wild type Pentin cDNA were obtained and assayed for increased lipid acyl hydrolase activity.

Cloning of products

Plasmid pMAXY 2021 was created from plasmid pMAL-c2x (New England Biolabs) in the following manner: pMAL-c2x was digested with Nde I and Xba I, treated with alkaline phosphatase (New England Biolabs) and purified by agarose gel electrophoresis. The portion of the wild type Pentin coding sequence encoding the processed form of Pentin was amplified by PCR using oligos BC24 and BC33 (SEQ ID NO:44 and SEQ ID NO:45, respectively). The resulting PCR products were digested with Nde I and Xba I and cloned into the purified, digested pMAL-c2x vector to yield pMAXY 2021. pMAXY 2021 was digested with BamH I and Xba I, treated with alkaline phosphatase, and purified by agarose gel electrophoresis.

Library polynucleotides were amplified by PCR, digested with BamH I and Xba I, and purified by agarose gel electrophoresis. The digested libraries were then ligated to plasmid pMAXY 2021 (see below), and transformed into E. coli. Clones were grown in the 2xYT medium containing 100 μg/ml carbenicillin to select for plasmid maintenance, and 1% glucose to maintain repression of the modified Lac promoter. Plasmid pMAXY 2045 is a slight variant of pMAXY 2021, generated in a similar fashion, using oligos BC34 and BC35 (SEQ ID NO:46 and SEQ ID NO:47, respectively). The organization of pMAXY 2021 and pMAXY 2045 are such that the wild type and genes from the library were cloned downstream of the promoter, properly distanced from a strong ribosome binding site, and in frame with an ATG codon six nucleotides upstream of the wild type or the library polynucleotides coding sequence. Consequently, pMAXY 2045 generated translation products containing a methionine-glycine-phenylalanine tripeptide at the N-terminus, while pMAXY 2021 and all library genes generated translation products containing a methionine-glycine-serine tri-peptide at the N-terminus.

### Example 2

### Screen for active Pentin clones

A screen was developed that allowed for the identification of clones with acyl hydrolase activity. This method involved plating recombinant libraries on square 23×23 cm trays (Genetix), and picking individual colonies using a Q Bot colony picking robot. Colonies were picked into 96 well or 384 well plates containing suitable growth medium and an antibiotic to select for plasmid maintenance. These plates were grown to stationary phase at 37 C, then stamped to duplicate plates (A, and B).

Plate A contained standard agar media plus antibiotic, while plate B contained standard agar media, antibiotic, and IPTG, to induce production of recombinant protein. After incubation to yield colonies, plate B was overlaid with a molten solution of 0.6% agarose, 75% N,N-Dimethyl formamide, 1.6 mg/ml a-napthyl caproate, 160 μg/ml α-napthyl caprylate, 40 μg/ml α-napthyl caprate, and 1.3 mg/ml Fast Blue RR salt. Alternatively, concentrations of substrates were varied to 160 μg/ml α-napthyl caproate, 160 μg/ml α-napthyl caprylate, 320 μg/ml α-napthyl caprate. Colonies having acyl hydrolase activity upon the caproate, caprylate or caprate substrates were identified by formation of a brown precipitate. Plate B was imaged using a digital video camera attached to a Q Bot robot. After imaging, plate B was exchanged for plate A, and the Q Bot picked individual colonies representing the replicas of active clones. These clones were picked individually into 96 well plates, and grown to stationary phase at 37 C.

### Example 3

### Identification of improved lipid acyl hydrolases

### Sample Preparation

Samples were grown to yield 2 ml of induced culture in a two-step process. Typically, 25 μl from each saturated culture was inoculated into 200 μl growth medium in 48 well deep well plates (Polyfiltronics), and incubated at 37 C until saturation. At this point, 1.8 ml media was added, and the cultures shaken at 250 rpm at 18 C for 1 hr. At this stage cultures typically had an optical density reading (O.D.) of ≠0.9. Next, cultures were induced by addition of IPTG (to 1 mM), covered with Airpore filters, and shaken at 250 rpm, 16 C for 24 hours. At the end of induction cultures were chilled to 4 C, centrifuged, media removed, and cultures frozen at −20 C. Cultures were then lysed by addition of 300 μl BPER lysis reagent (Pierce) supplemented with DNAse to 2 U/ml, Mg⁺⁺ to 5 mM, and lysozyme to 0.2 mg/ml. Cultures were shaken for 30 minutes at room temperature, and then frozen at −20 C. Clones were grown in duplicate.

### Assay for Improved Acyl Hydrolase Activity.

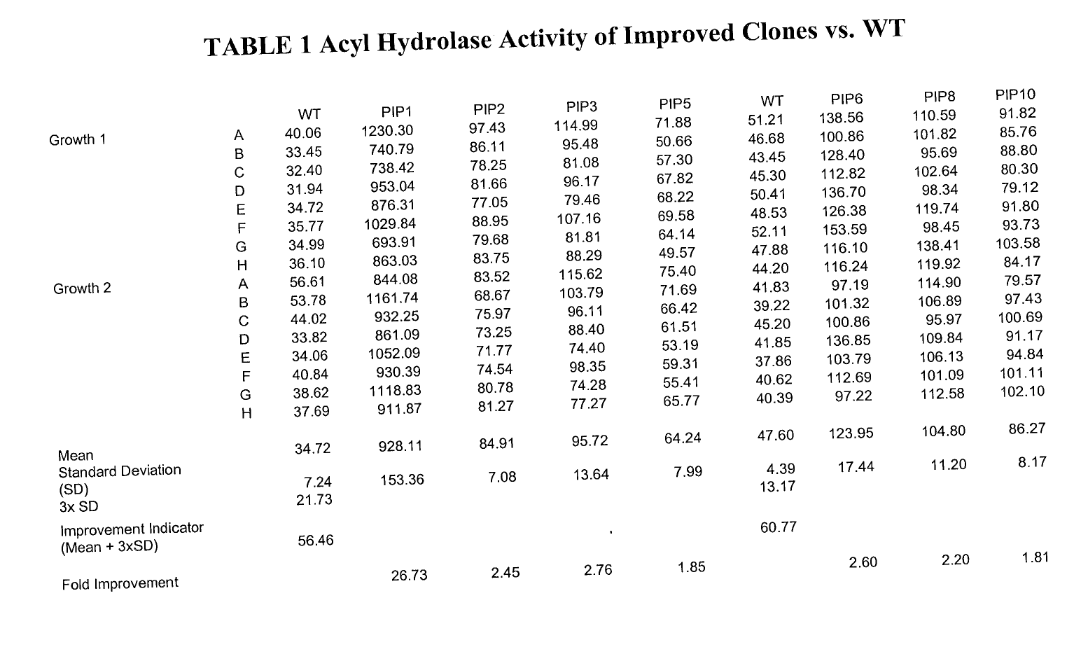

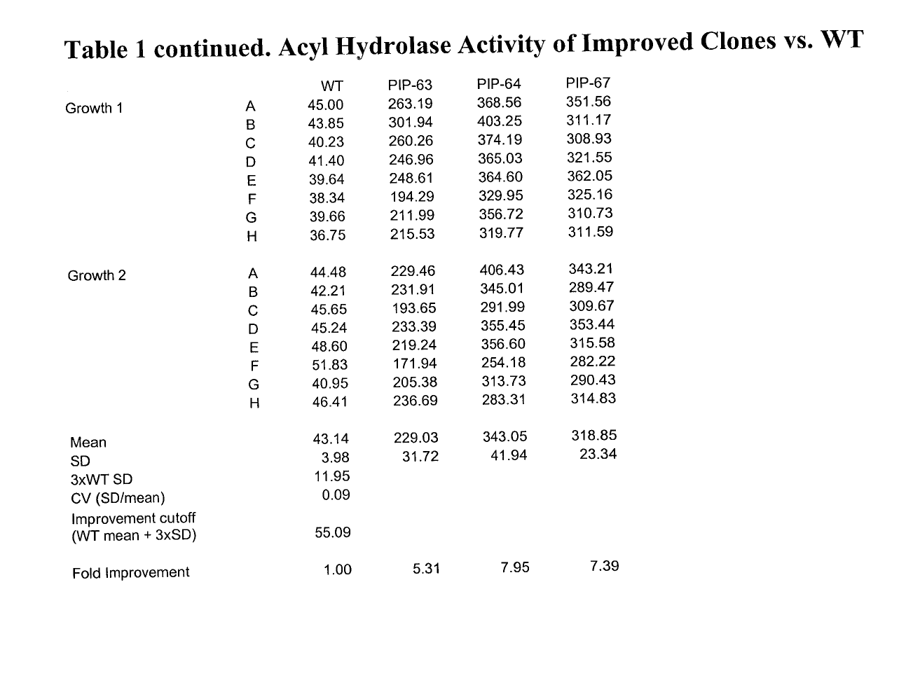

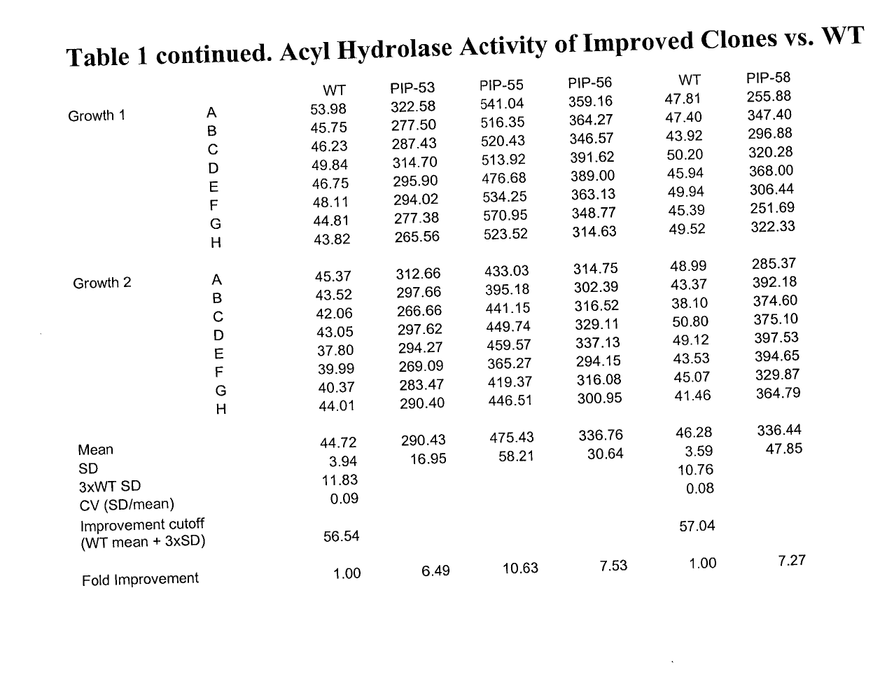

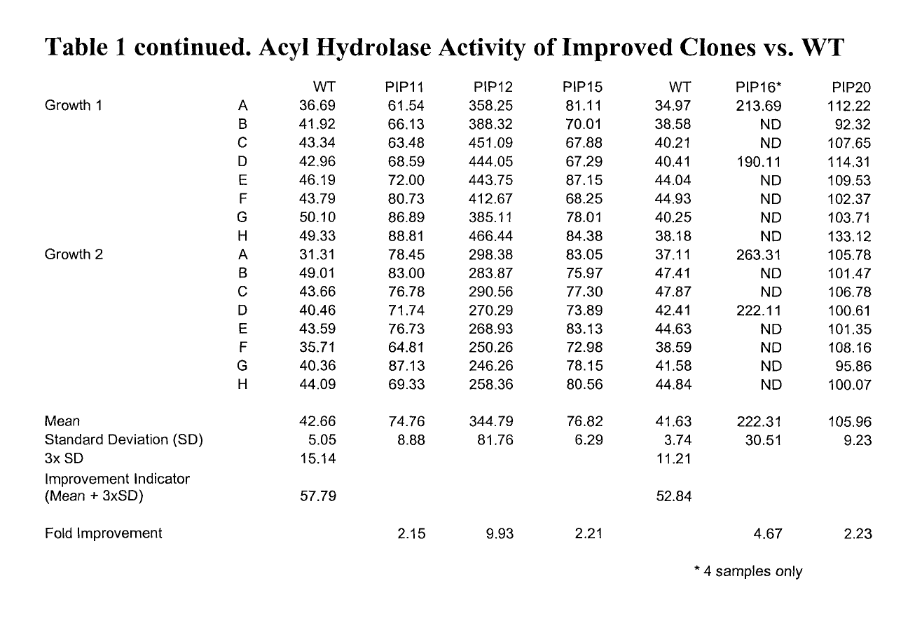

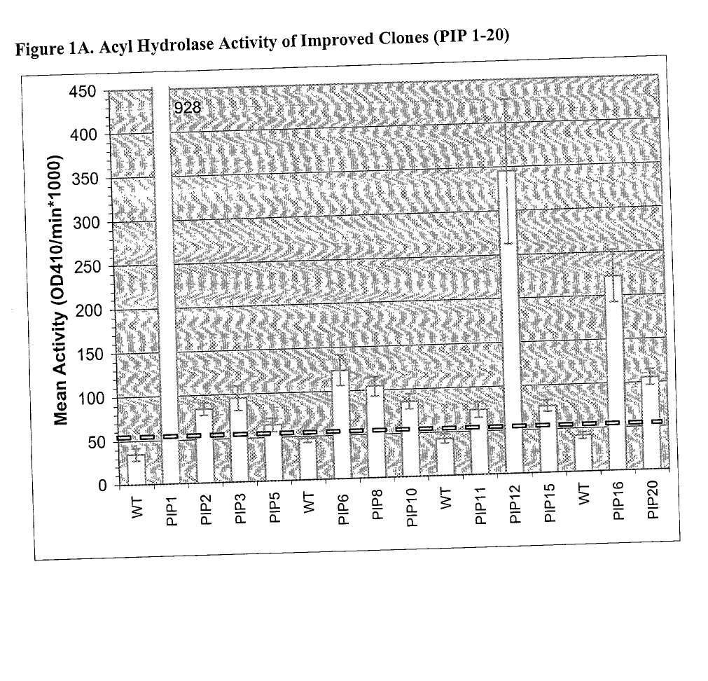

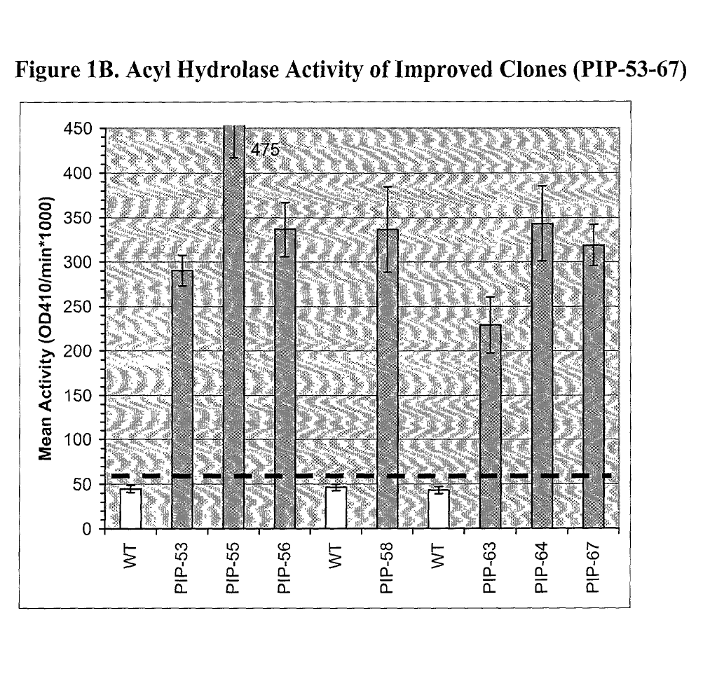

Clones were assayed for ability to hydrolyze p-nitrophenyl caprylate, a soluble fatty acid substrate. Typically, 10 μl of crude lysate was added to 155 μl buffer (100 mM Tris, pH 8.2, 100 mM KCl) in 96 well plate. The assay was commenced by addition of 35 μl substrate (p-Nitrophenyl Caprylate (Sigma) 4 mM in 1% Triton X-100, 50 mM KCl, 100 mM Tris, pH 8.2). After mixing, the change in OD at 410 nm was recorded using a Spectramax U-VNIS spectrophotometer. Both induced cultures of wild type Pentin in pMAXY2045 and pMAXY2045 alone served as controls. Average activity, standard deviation, and coefficient of variance (CV) were determined for wild type clones, and clones showing activity three standard deviations greater than wild type were selected for re-testing. Samples were re-grown and lysed as before, typically picking eight individual colonies per clone. Wild type controls were grown in the same plate (typically eight colonies/plate, in duplicate plates) to serve as controls. Lysates from clones comprising the empty vector served as negative controls. The acyl hydrolase activity of each improved clone relative to wild type is shown in Table 1 and FIG. 1A and 1B. Clones with activity consistently greater than 1.5X wild type were selected.

In the initial library, we screened approximately 2,100 clones. Of these 2,100 clones, 288 clones were chosen as active in the screen assay, and assayed for improved acyl hydrolase activity. Clones PIP-1, 2, 3, 5, 6, 8, 10, 11, 12, 15, 16, 20 showed activity much greater than wild type in both the initial assay and upon re-testing. Assay of 10,600 additional clones yielded approximately 2,000 additional active clones. Assay of these active clones yielded large numbers of improved clones. Those having activity greater than 5x wild type activity upon re-growth and re-testing (usually 8 colonies per plate, in duplicate plates) were chosen for DNA sequencing. These clones are designated PIP-53, PIP-55, PIP-56, PIP-57, PIP-58, PIP-62, PIP-63, PIP-64, and PIP-67.

### Analysis of Protein Expression by PAGE and Western Blot

Analysis of several improved clones by PAGE and Western Blot showed that improvements in activity were not due to increased expression. No detectable difference was seen in the level of Pentin produced in improved clones vs. wild type controls, or relative to non-improved clones from the same plates.

### Kinetic Analysis of Improved Clones

Initial kinetic assay of the highest activity clone (PIP-1) indicates that improvement results from increased Vₘₐₓ, and not a lowering of km. This suggests that improvement in K_{cat} (i.e., release of the cleaved fatty acid) is the step affected, and is likely to be the rate-limiting step in the enzymatic reaction.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. An isolated nucleic acid comprising a polynucleotide encoding a polypeptide at least 70% identical to a polypeptide selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38 and SEQ ID NO:40, with the proviso that the polypeptide is not SEQ ID NO:42 or SEQ ID NO:43,
wherein the polypeptide exhibits lipid acyl hydrolase activity.

2. The isolated nucleic acid of claim 1, wherein the polypeptide exhibits a lipid acyl hydrolase activity at least 20% of the lipid acyl hydrolase activity of SEQ ID NO:41.

3. The isolated nucleic acid of claim 1, wherein the polypeptide exhibits a lipid acyl hydrolase activity at least 200% of the lipid acyl hydrolase activity of SEQ ID NO:41.

4. The isolated nucleic acid of claim 1, wherein the polypeptide exhibits lipid acyl hydrolase activity at least 1,000% of the lipid acyl hydrolase activity of SEQ ID NO:41.

5. The isolated nucleic acid of claim 1, wherein the polynucleotide comprises SEQ ID NO:1.

6. The isolated nucleic acid of claim 1, wherein the polynucleotide comprises SEQ ID NO:3.

7. The isolated nucleic acid of claim 1, wherein the polynucleotide comprises SEQ ID NO:5.

8. The isolated nucleic acid of claim 1, wherein the polynucleotide comprises SEQ ID NO:7.

9. The isolated nucleic acid of claim 1, wherein the polynucleotide comprises SEQ ID NO:9.

10. The isolated nucleic acid of claim 1, wherein the polynucleotide comprises SEQ ID NO:11.

11. The isolated nucleic acid of claim 1, wherein the polynucleotide comprises SEQ ID NO:13.

12. The isolated nucleic acid of claim 1, wherein the polynucleotide comprises SEQ ID NO:15.

13. The isolated nucleic acid of claim 1, wherein the polynucleotide comprises SEQ ID NO:17.

14. The isolated nucleic acid of claim 1, wherein the polynucleotide comprises SEQ ID NO:19.

15. The isolated nucleic acid of claim 1, wherein the polynucleotide comprises SEQ ID NO:21.

16. The isolated nucleic acid of claim 1, wherein the polynucleotide comprises SEQ ID NO:23.

17. The isolated nucleic acid of claim 1, wherein the polynucleotide comprises SEQ ID NO:25.

18. The isolated nucleic acid of claim 1, wherein the polynucleotide comprises SEQ ID NO:27.

19. The isolated nucleic acid of claim 1, wherein the polynucleotide comprises SEQ ID NO:29.

20. The isolated nucleic acid of claim 1, wherein the polynucleotide comprises SEQ ID NO:31.

21. The isolated nucleic acid of claim 1, wherein the polynucleotide comprises SEQ ID NO:33.

22. The isolated nucleic acid of claim 1, wherein the polynucleotide comprises SEQ ID NO:35.

23. The isolated nucleic acid of claim 1, wherein the polynucleotide comprises SEQ ID NO:37.

24. The isolated nucleic acid of claim 1, wherein the polynucleotide comprises SEQ ID NO:39.

25. The isolated nucleic acid of claim 1, wherein the polypeptide comprises SEQ ID NO:2.

26. The isolated nucleic acid of claim 1, wherein the polypeptide comprises SEQ ID NO:4.

27. The isolated nucleic acid of claim 1, wherein the polypeptide comprises SEQ ID NO:6.

28. The isolated nucleic acid of claim 1, wherein the polypeptide comprises SEQ ID NO:8.

29. The isolated nucleic acid of claim 1, wherein the polypeptide comprises SEQ ID NO:10.

30. The isolated nucleic acid of claim 1, wherein the polypeptide comprises SEQ ID NO:12.

31. The isolated nucleic acid of claim 1, wherein the polypeptide comprises SEQ ID NO:14.

32. The isolated nucleic acid of claim 1, wherein the polypeptide comprises SEQ ID NO:16.

33. The isolated nucleic acid of claim 1, wherein the polypeptide comprises s SEQ ID NO:18.

34. The isolated nucleic acid of claim 1, wherein the polypeptide comprises SEQ ID NO:20.

35. The isolated nucleic acid of claim 1, wherein the polypeptide comprises SEQ ID NO:22.

36. The isolated nucleic acid of claim 1, wherein the polypeptide comprises SEQ ID NO:24.

37. The isolated nucleic acid of claim 1, wherein the polypeptide comprises SEQ ID NO:26.

38. The isolated nucleic acid of claim 1, wherein the polypeptide comprises SEQ ID NO:28.

39. The isolated nucleic acid of claim 1, wherein the polypeptide comprises SEQ ID NO:30.

40. The isolated nucleic acid of claim 1, wherein the polypeptide comprises SEQ ID NO:32.

41. The isolated nucleic acid of claim 1, wherein the polypeptide comprises SEQ ID NO:34.

42. The isolated nucleic acid of claim 1, wherein the polypeptide comprises SEQ ID NO:36.

43. The isolated nucleic acid of claim 1, wherein the polypeptide comprises SEQ ID NO:38.

44. The isolated nucleic acid of claim 1, wherein the polypeptide comprises SEQ ID NO:40.

45. The isolated nucleic acid of claim 1, further comprising a promoter operably linked to the polynucleotide.

46. The isolated nucleic acid of claim 45, wherein the promoter is a tissue-preferred promoter.

47. The isolated nucleic acid of claim 45, wherein the promoter is a constitutive promoter.

48. The isolated nucleic acid of claim 45, wherein the promoter is an inducible promoter.

49. A vector comprising the nucleic acid of claim 45.

50. The vector of claim 49, wherein the promoter is a tissue-preferred promoter.

51. The vector of claim 49, wherein the promoter is a constitutive promoter.

52. The vector of claim 49, wherein the promoter is an inducible promoter.

53. An isolated nucleic acid comprising a polynucleotide encoding a polypeptide at least 70% identical to a polypeptide selected from the group consisting of SEQ ID NO:2, SEQ ID NO:4, SEQ ID NO:6, SEQ ID NO:8, SEQ ID NO:10, SEQ ID NO:12, SEQ ID NO:1 4, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38 and SEQ ID NO:40, with the proviso that the polypeptide is not SEQ ID NO:42 or SEQ ID NO:43,
wherein the polypeptide exhibits insecticidal activity.

54. An isolated nucleic acid comprising a polynucleotide encoding a polypeptide with lipid acyl hydrolase or insecticidal activity, wherein the nucleic acid specifically hybridizes under stringent conditions to a probe polynucleotide selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37 and SEQ ID NO:39, with the proviso that the polynucleotide does not encode SEQ ID NO:42 or SEQ ID NO:43.

55. The isolated nucleic acid of claim 54, wherein the polypeptide exhibits lipid acyl hydrolase activity.

56. The isolated nucleic acid of claim 54, wherein the polypeptide exhibits insecticidal activity.

57. The isolated nucleic acid of claim 54, wherein the polypeptide exhibits a lipid acyl hydrolase activity at least 20% of the lipid acyl hydrolase activity of SEQ ID NO:41.

58. The isolated nucleic acid of claim 54, wherein the polypeptide exhibits a lipid acyl hydrolase activity at least 200% of the lipid acyl hydrolase activity of SEQ ID NO:41.

59. The isolated nucleic acid of claim 54, wherein the polypeptide exhibits a lipid acyl hydrolase activity at least 1,000% of the lipid acyl hydrolase activity of SEQ ID NO:41.

60. The isolated nucleic acid of claim 54, wherein the probe polynucleotide comprises SEQ ID NO:1.

61. The isolated nucleic acid of claim 54, wherein the probe polynucleotide comprises SEQ ID NO:3.

62. The isolated nucleic acid of claim 54, wherein the probe polynucleotide comprises SEQ ID NO:5.

63. The isolated nucleic acid of claim 54, wherein the probe polynucleotide comprises SEQ ID NO:7.

64. The isolated nucleic acid of claim 54, wherein the probe polynucleotide comprises SEQ ID NO:9.

65. The isolated nucleic acid of claim 54, wherein the probe polynucleotide comprises SEQ ID NO:11.

66. The isolated nucleic acid of claim 54, wherein the probe polynucleotide comprises SEQ ID NO:13.

67. The isolated nucleic acid of claim 54, wherein the probe polynucleotide comprises SEQ ID NO:15.

68. The isolated nucleic acid of claim 54, wherein the probe polynucleotide comprises SEQ ID NO:17.

69. The isolated nucleic acid of claim 54, wherein the probe polynucleotide comprises SEQ ID NO:19.

70. The isolated nucleic acid of claim 54, wherein the probe polynucleotide comprises SEQ ID NO:21.

71. The isolated nucleic acid of claim 54, wherein the probe polynucleotide comprises SEQ ID NO:23.

72. The isolated nucleic acid of claim 54, wherein the probe polynucleotide comprises SEQ ID NO:25.

73. The isolated nucleic acid of claim 54, wherein the probe polynucleotide comprises SEQ ID NO:27.

74. The isolated nucleic acid of claim 54, wherein the probe polynucleotide comprises SEQ ID NO:29.

75. The isolated nucleic acid of claim 54, wherein the probe polynucleotide comprises SEQ ID NO:31.

76. The isolated nucleic acid of claim 54, wherein the probe polynucleotide comprises SEQ ID NO:33.

77. The isolated nucleic acid of claim 54, wherein the probe polynucleotide comprises SEQ ID NO:35.

78. The isolated nucleic acid of claim 54, wherein the probe polynucleotide comprises SEQ ID NO:37.

79. The isolated nucleic acid of claim 54, wherein the probe polynucleotide comprises SEQ ID NO:39.

80. The isolated nucleic acid of claim 54, further comprising a promoter operably linked to the polynucleotide.

81. The isolated nucleic acid of claim 80, wherein the promoter is a tissue-preferred promoter

82. The isolated nucleic acid of claim 80, wherein the promoter is a constitutive promoter.

83. The isolated nucleic acid of claim 80, wherein the promoter is an inducible promoter.

84. An isolated nucleic acid comprising a polynucleotide encoding a polypeptide with lipid acyl hydrolase or insecticidal activity, wherein the polypeptide is at least 70% identical to SEQ ID NO:41 and the polypeptide comprises at least one of the following alterations: D240G, I241T, I241N or S49P.

85. The isolated nucleic acid of claim 84, wherein the polypeptide exhibits lipid acyl hydrolase activity.

86. The isolated nucleic acid of claim 84, wherein the polypeptide exhibits insecticidal activity.

87. An isolated nucleic acid of at least 20 nucleotides in length, the nucleic acid encoding an amino acid subsequence of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38 and SEQ ID NO:40, with the proviso that the nucleic acid does not encode an amino acid subsequence of SEQ ID NO:42 or SEQ ID NO:43.

88. The isolated nucleic acid of claim 87, wherein the encoded amino acid sequence exhibits lipid acyl hydrolase or insecticidal activity.

89. The isolated nucleic acid of claim 88, wherein the encoded amino acid sequence exhibits improved lipid acyl hydrolase activity compared to the lipid acyl hydrolase activity of SEQ ID NO:41.

90. An isolated polyp eptide comprising at least 70% identity to a polypeptide selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:11, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38 and SEQ ID NO:40, with the proviso that the polypeptide is not SEQ ID NO:42 or SEQ ID NO:43,
wherein the isolated polypeptide exhibits lipid acyl hydrolase or insecticidal activity.

91. The isolated polypeptide of claim 90, wherein the isolated polypeptide exhibits lipid acyl hydrolase activity.

92. The isolated polypeptide of claim 90, wherein the isolated polypeptide exhibits insecticidal activity.

93. The isolated polypeptide of claim 90, wherein the isolated polypeptide exhibits a lipid acyl hydrolase activity at least 20% of the lipid acyl hydrolase activity of SEQ ID NO:41.

94. The isolated polypeptide of claim 90, wherein the polypeptide exhibits a lipid acyl hydrolase activity at least 200% of the lipid acyl hydrolase activity of SEQ ID NO:41.

95. The isolated polypeptide of claim 90, wherein the polypeptide exhibits a lipid acyl hydrolase activity at least 1,000% of the lipid acyl hydrolase activity of SEQ ID NO:41.

96. The isolated polypeptide of claim 90, wherein the polypeptide comprises SEQ ID NO:4.

97. The isolated polypeptide of claim 90, wherein the polypeptide comprises SEQ ID NO:6.

98. The isolated polypeptide of claim 90, wherein the polypeptide comprises SEQ ID NO:8.

99. The isolated polypeptide of claim 90, wherein the polypeptide comprises SEQ ID NO:10.

100. The isolated polypeptide of claim 90, wherein the polypeptide comprises SEQ ID NO:12.

101. The isolated polypeptide of claim 90, wherein the polypeptide comprises SEQ ID NO:14.

102. The isolated polypeptide of claim 90, wherein the polypeptide comprises SEQ ID NO:16.

103. The isolated polypeptide of claim 90, wherein the polypeptide comprises SEQ ID NO:18.

104. The isolated polypeptide of claim 90, wherein the polypeptide comprises SEQ ID NO:20.

105. The isolated polypeptide of claim 90, wherein the polypeptide comprises SEQ ID NO:22.

106. The isolated polypeptide of claim 90, wherein the polypeptide comprises SEQ ID NO:24.

107. The isolated polypeptide of claim 90, wherein the polypeptide comprises SEQ ID NO:26.

108. The isolated polypeptide of claim 90, wherein the polypeptide comprises SEQ ID NO:28.

109. The isolated polypeptide of claim 90, wherein the polypeptide comprises SEQ ID NO:30.

110. The isolated polypeptide of claim 90, wherein the polypeptide comprises SEQ ID NO:32.

111. The isolated polypeptide of claim 90, wherein the polypeptide comprises SEQ ID NO:34.

112. The isolated polypeptide of claim 90, wherein the polypeptide comprises SEQ ID NO:36.

113. The isolated polypeptide of claim 90, wherein the polypeptide comprises SEQ ID NO:38.

114. The isolated polypeptide of claim 90, wherein the polypeptide comprises SEQ ID NO:40.

115. An isolated polynucleotide with lipid acyl hydrolase or insecticidal activity, wherein the polypeptide is at least 70% identical to SEQ ID NO:41 and the polypeptide comprises at least one of the following alterations: D240G, I241T, I241N or S49P.

116. An antibody capable of binding the isolated polypeptide of claim 90.

117. A plant comprising a recombinant expression cassette comprising a promoter operably linked to a polynucleotide encoding a polypeptide with lipid acyl hydrolase or insecticidal activity, wherein the polypeptide is at least 70% identical to a polypeptide elected from the group comprising SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38 and SEQ ID NO:40, with the proviso that the polypeptide is not SEQ ID NO:42 or SEQ ID NO:43.

118. The plant of claim 117, wherein the polypeptide exhibits lipid acyl hydrolase activity.

119. The plant of claim 117, wherein the polypeptide insecticidal activity.

120. The plant of claim 117, wherein the polypeptide exhibits a lipid acyl hydrolase activity at least 20% of the lipid acyl hydrolase activity of SEQ ID NO:41.

121. The plant of claim 117, wherein the polypeptide exhibits a lipid acyl hydrolase activity at least 200% of the lipid acyl hydrolase activity of SEQ ID NO:41.

122. The plant of claim 117, wherein the polypeptide exhibits a lipid acyl hydrolase activity at least 1,000% of the lipid acyl hydrolase activity of SEQ ID NO:41.

123. The plant of claim 117, wherein the polypeptide comprises SEQ ID NO:2.

124. The plant of claim 117, wherein the polypeptide comprises SEQ ID NO:4.

125. The plant of claim 117, wherein the polypeptide comprises SEQ ID NO:6.

126. The plant of claim 117, wherein the polypeptide comprises SEQ ID NO:8.

127. The plant of claim 117, wherein the polypeptide comprises SEQ ID NO:10.

128. The plant of claim 117, wherein the polypeptide comprises SEQ ID NO:12.

129. The plant of claim 117, wherein the polypeptide comprises SEQ ID NO:14.

130. The plant of claim 117, wherein the polypeptide comprises SEQ ID NO:16.

131. The plant of claim 117, wherein the polypeptide comprises SEQ ID NO:18.

132. The plant of claim 117, wherein the polypeptide comprises SEQ ID NO:20.

133. The plant of claim 117, wherein the polypeptide comprises SEQ ID NO:22.

134. The plant of claim 117, wherein the polypeptide comprises SEQ ID NO:24.

135. The plant of claim 117, wherein the polypeptide comprises SEQ ID NO:26.

136. The plant of claim 117, wherein the polypeptide comprises SEQ ID NO:28.

137. The plant of claim 117, wherein the polypeptide comprises SEQ ID NO:30.

138. The plant of claim 117, wherein the polypeptide comprises SEQ ID NO:32.

139. The plant of claim 117, wherein the polypeptide comprises SEQ ID NO:34.

140. The plant of claim 117, wherein the polypeptide comprises SEQ ID NO:36.

141. The plant of claim 117, wherein the polypeptide comprises SEQ ID NO:38.

142. The plant of claim 117, wherein the polypeptide comprises SEQ ID NO:40.

143. The plant of claim 117, wherein the promoter is constitutive.

144. The plant of claim 117, wherein the promoter is tissue-preferred.

145. The plant of claim 117, wherein the promoter is inducible.

146. The plant of claim 117, wherein the plant is selected from the group consisting of maize, soybean, potato and cotton.

147. A plant comprising a polynucleotide encoding a polypeptide with lipid acyl hydrolase or insecticidal activity, wherein the polypeptide is at least 70% identical to SEQ ID NO:41 and the polypeptide comprises at least one of the following alterations: D240G, I241T, I241N or S49P.

148. A method of enhancing plant resistance to a pest, the method comprising
a) introducing into the plant a recombinant expression cassette comprising a promoter operably linked to a polynucleotide encoding a polypeptide at least 70% identical to a polypeptide selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38 and SEQ ID NO:40, with the proviso that the polypeptide is not SEQ ID NO:42 or SEQ ID NO:43, and
b) selecting a plant with enhanced resistance.

149. The method of claim 148, wherein the plant is selected from the group consisting of maize, soybean, potato and cotton.

150. The method of claim 148, wherein the polypeptide comprises SEQ ID NO:2.

151. The method of claim 148, wherein the polypeptide comprises SEQ ID NO:4.

152. The method of claim 148, wherein the polypeptide comprises SEQ ID NO:6.

153. The method of claim 148, wherein the polypeptide comprises SEQ ID NO:8.

154. The method of claim 148, wherein the polypeptide comprises SEQ ID NO:10.

155. The method of claim 148, wherein the polypeptide comprises SEQ ID NO:12.

156. The method of claim 148, wherein the polypeptide comprises SEQ ID NO:14.

157. The method of claim 148, wherein the polypeptide comprises SEQ ID NO:16.

158. The method of claim 148, wherein the polypeptide comprises SEQ ID NO:18.

159. The method of claim 148, wherein the polypeptide comprises SEQ ID NO:20.

160. The method of claim 148, wherein the polypeptide comprises SEQ ID NO:22.

161. The method of claim 148, wherein the polyp eptide comprises SEQ ID NO:24.

162. The method of claim 148, wherein the polypeptide comprises SEQ ID NO:26.

163. The method of claim 148, wherein the polypeptide comprises SEQ ID NO:28.

164. The method of claim 148, wherein the polypeptide comprises SEQ ID NO:30.

165. The method of claim 148, wherein the polypeptide comprises SEQ ID NO:32.

166. The method of claim 148, wherein the polypeptide comprises SEQ ID NO:34.

167. The method of claim 148, wherein the polypeptide comprises SEQ ID NO:36.

168. The method of claim 148, wherein the polypeptide comprises SEQ ID NO:38.

169. The method of claim 148, wherein the polypeptide comprises SEQ ID NO:40.

170. The method of claim 148, wherein the promoter is constitutive.

171. The method of claim 148, wherein the promoter is tissue-preferred.

172. The method of claim 148, wherein the promoter is inducible.

173. A method of enhancing plant resistance to a pest, the method comprising
a) introducing into the plant a recombinant expression cassette comprising a promoter operably linked to a polynucleotide encoding a polypeptide with lipid acyl hydrolase or insecticidal activity, wherein the polypeptide is at least 70% identical to SEQ ID NO:41 and the polypeptide comprises at least one of the following alterations: D240G, I241T, I241N or S49P; and
b) selecting a plant with enhanced resistance.
